(19) United States
(12) Patent Application Publication
(10) Pub. No.: US 2021/0317220 A1
(43) Pub. Date: Oct. 14, 2021

(54) METHOD OF PROVIDING SAFE ADMINISTRATION OF AN ANTI-CD154 ANTIBODY

(71) Applicant: Janssen Biotech, Inc., Horsham, PA (US)

(72) Inventors: Mark RIGBY, Jenkintown, PA (US); Donald RAIBLE, Berwyn, PA (US); Alexa PIANTONE, Pottstown, PA (US); Jocelyn LEU, Ambler, PA (US); Nina Chi Sabins, Plymouth, PA (US)

(73) Assignee: Janssen Biotech, Inc., Horsham, PA (US)

(21) Appl. No.: 17/274,512

(22) PCT Filed: Sep. 24, 2019
(86) PCT No.: PCT/US2019/052558

§ 371 Date: Mar. 9, 2021

Related U.S. Application Data
(60) Provisional application No. 62/826,131, filed on Mar. 29, 2019.
(60) Provisional application No. 62/735,529, filed on Sep. 24, 2018.

(51) Int. Cl. C07K 16/28; A61K 47/26; A61K 47/18

(52) U.S. Cl. CPC C07K 16/2875; A61K 2039/505; A61K 47/183; A61K 47/26

(57) ABSTRACT

Methods for clinically proven safe administration of an anti-CD 154 antibody or antigen binding fragment thereof by subcutaneous or intravenous administration are provided. Also provided are methods for clinically proven safe treatment of an autoimmune disease by subcutaneous or intravenous administration of an anti-CD 154 antibody or antigen binding fragment thereof, such as rheumatoid arthritis, systemic lupus erythematosus (SLE), and Sjögren's Syndrome.

### CROSS REFERENCE TO RELATED APPLICATIONS

This application claims priority under 35 U.S.C. § 119(e) to U.S. Provisional Application No. 62/735,529, filed Sep. 24, 2018, and U.S. Provisional Application No. 62/826,131, filed Mar. 29, 2019, the disclosures of which are herein incorporated by reference in their entireties.

### REFERENCE TO SEQUENCE LISTING SUBMITTED ELECTRONICALLY

This application contains a sequence listing, which is submitted electronically via EFS-Web as an ASCII formatted sequence listing with a file name “688097_525_Sequence Listing,” creation date of Sep. 13, 2019, and having a size of 12.8 kb. The sequence listing submitted via EFS-Web is part of the specification and is herein incorporated by reference in its entirety.

### FIELD OF THE INVENTION

The invention relates to methods of providing clinically proven safe subcutaneous and intravenous administration of an anti-CD154 antibody, and methods of providing clinically proven safe treatment of an autoimmune disease by subcutaneous or intravenous administration of an anti-CD154 antibody.

### BACKGROUND OF THE INVENTION

CD154, also known as CD40 ligand (CD40L), gp39, TNF-related activation protein (TRAP), or anti-T cell/B cell activating molecule (T-BAM), is a trimeric transmembrane protein of the tumor necrosis factor (TNF) superfamily. Human CD154 is found both on the cell membrane as a type II membrane protein and also exists in soluble form in plasma. CD154 is expressed in an activation-dependent, temporally-restricted manner on the surface of CD4⁺ T cells. CD154 is also expressed, following activation, on a subset of CD8⁺ T cells, basophils, mast cells, eosinophils, natural killer cells, B cells, macrophages, dendritic cells and platelets. CD154 also exists as a soluble form in the blood.

CD154 binds to CD40 on antigen-presenting cells (APC), which leads to various responses depending on the target cell type. The CD40-CD154 interaction is essential for normal T-B cell interactions, including increased co-stimulation, T-cell priming, cytokine production, antibody-class switching and affinity maturation, and antibody and autoantibody production.

Disruption of the CD40/CD154 pathway via CD154 blockage has been shown to be beneficial in autoimmune diseases such as systemic lupus erythematosus (SLE), rheumatoid arthritis (RA), multiple sclerosis (MS), inflammatory bowel disease (IBD), type I diabetes (T1D), and allograft rejection. In humans, mutations in either CD40 or CD154 result in hyper-IgM syndrome characterized by lack of IgG or IgA isotypes (Aruffo et al., Cell 72:291, 1993).

Anti-CD154 antibodies have been described for example in Int. Pat. Publ. Nos. WO1993/08207, WO1994/10308, WO1996/40918, WO1993/009812, WO1999/051258, WO1995/006480, WO1995/006481, WO1995/006666, WO2001/002057, WO1997/017446, WO1999/012566, WO2001/068860, WO2005/003175, WO2006/033702, WO2006/030220, WO2008/118356, WO2012/052205, WO2012/138768, WO2012/138768, WO2013/055745, WO2013/056068, and WO2017/024146.

Anti-CD154 antibodies have been shown to be efficacious in the treatment of autoimmune diseases in humans. However, thromboembolism due to platelet activation observed upon treatment prohibited continued clinical development. Engagement of FcγRIIa on platelets has been shown to be causative for platelet activation by the anti-CD154 antibody 5c8 (Xie et al., J Immunol 192:4083-4092, 2014). Further, early-phase clinical studies of previous formulations of anti-CD154 antibodies resulted in unexpected thromboembolic events (TEs) in subjects administered such antibodies, including myocardial infarction (MI), pulmonary embolism (PE), and deep vein thrombosis (DVT).

For example, in the late 1990s and early 2000s, anti-CD154 mAbs were investigated in human auto- and allo-immunity, including SLE, immune thrombocytopenia purpura, Crohn's disease, and renal transplant (e.g., Boumpas et al. Arthritis Rheum. 48(3): 719-727, 2003; Couzin, Science 307; 1712-1715, 2005; Davis et al. J. Rheumat. 28: 95-101, 2001; Dumont, Current Opinion in Investigational Drugs, 3(5): 725-734, 2002; Elgueta et al. Immunological Reviews, 229(1): 152-172, 2009; Kalunian et al. Arthritis and Rheumatism, 46(12): 3251-3258, 2002; Kuwana et al. Blood, 103(4): 1229-1236, 2004; Patel et al. BJH, 141:545-548, 2007; Yazdany, Lupus, 12:377-380, 2004). Development was halted due to unexpected observations of thromboembolic events in some subjects, although there is limited data regarding the specifics of these thromboembolic events, including their timing in relation to drug dosing as well as the dosage and dosing regimen. These TE events are believed to be the result of platelet activation secondary to mAb:CD154 complexes binding to the Fc receptor, FcγRIIa, found on platelets.

Thus, subsequent anti-CD154 antibodies have been engineered to prevent the interaction of the antibody with FcγRIIa on platelets. See, e.g., WO2017/024146.

However, there is a need to determine a dosage of such anti-CD154 antibodies that can be safely administered, particularly via the subcutaneous or intravenous route, to subjects diagnosed with or suspected of having an autoimmune disorder, such as rheumatoid arthritis (RA), systemic lupus erythematosus (SLE), or Sjögren's Syndrome for safe treatment of such disorders.

### BRIEF SUMMARY OF THE INVENTION

The invention relates to the clinically proven safe administration of an anti-CD154 antibody to subjects, including for clinically proven safe treatment of an autoimmune disease in a subject, such as rheumatoid arthritis, systemic lupus erythematosus (SLE), or Sjögren's Syndrome.

In one general aspect, the invention relates to a method of providing clinically proven safe administration of an anti-CD154 antibody or antigen binding fragment thereof comprising heavy chain complementarity determining regions (CDRs) HCDR1, HCDR2, and HCDR3 of SEQ ID NOs: 3, 4, and 5, respectively, and light chain CDRs LCDR1, LCDR2, and LCDR3, of SEQ ID NOs: 6, 7, and 8, respectively, to a human subject in need thereof, the method comprising subcutaneously or intravenously administering to the subject a pharmaceutical composition comprising the anti-CD154 antibody or antigen binding fragment thereof and a pharmaceutically acceptable carrier, wherein a total dosage of the anti-CD154 antibody or antigen binding fragment thereof administered is 0.3 mg/kg to 50 mg/kg body weight of the subject per administration.

In another general aspect, the invention relates to a method of providing clinically proven safe treatment of an autoimmune disease in a human subject in need thereof, the method comprising subcutaneously or intravenously administering to the subject a pharmaceutical composition comprising an anti-CD154 antibody or antigen binding fragment thereof comprising heavy chain complementarity determining regions (CDRs) HCDR1, HCDR2, and HCDR3 of SEQ ID NOs: 3, 4, and 5, respectively, and light chain CDRs LCDR1, LCDR2, and LCDR3, of SEQ ID NOs: 6, 7, and 8, respectively, and a pharmaceutically acceptable carrier, wherein a total dosage of the anti-CD154 antibody or antigen binding fragment thereof administered per administration is 0.3 mg/kg to 50 mg/kg body weight of the subject. Preferably, the autoimmune disease is selected from rheumatoid arthritis, systemic lupus erythematosus, and Sjögren's Syndrome.

In one embodiment, the total dosage of the anti-CD154 antibody or antigen binding fragment thereof administered per administration is 0.3 mg/kg, 1 mg/kg, 3 mg/kg, 10 mg/kg, 30 mg/kg or 50 mg/kg body weight of the subject.

In one embodiment, the pharmaceutical composition is administered subcutaneously. In such embodiments, a total dosage of the anti-CD154 antibody or antigen binding fragment thereof is administered in one, two, three, or four subcutaneous injections per administration.

In one embodiment, the pharmaceutical composition is administered intravenously.

In one embodiment, clinically proven safe administration of the anti-CD154 antibody or antigen binding fragment thereof and/or clinically proven safe treatment of an autoimmune disease comprises viremia of <10,000 copies of viral DNA of at least one virus selected from the group consisting of Epstein-Barr virus (EBV) and cytomegalovirus (CMV) per mL of sample, preferably blood, serum, or plasma, from the subject.

In one embodiment, clinically proven safe administration of the anti-CD154 antibody or antigen binding fragment thereof and/or clinically proven safe treatment of an autoimmune disease comprises an immune response comprising at least one of a recall response and primary immune response, preferably an immune response comprising a recall response to tetanus toxoid and a primary response to keyhole limpet hemocyanin (KLH).

In one embodiment, clinically proven safe administration of the anti-CD154 antibody or antigen binding fragment thereof and/or clinically proven safe treatment of an autoimmune disease does not result in any clinically apparent thromboembolic (TE) event in the subject.

In one embodiment, clinically proven safe administration of the anti-CD154 antibody or antigen binding fragment thereof and/or clinically proven safe treatment of an autoimmune disease does not result in activation of platelets.

In another embodiment, the pharmaceutical composition comprises 40 mg/mL to 60 mg/mL of the anti-CD154 antibody or antigen binding fragment thereof, 1 mM to 20 mM histidine, 5% to 10% (w/v) sucrose, 0.01% to 0.1% (w/v) polysorbate 20 (PS20), and 10 μg/mL to 30 μg/mL EDTA, at pH 5.0-6.0.

In another embodiment, the pharmaceutical composition comprises 40 mg/mL to 60 mg/mL of the anti-CD154 antibody or antigen binding fragment thereof, 1 mM to 20 mM arginine, 5% to 10% (w/v) lactose, 0.01% to 0.10% (w/v) polysorbate 80 (PS80), and 10 μg/mL to 30 μg/mL EDDS, at pH 5.0-6.0.

In another embodiment, the pharmaceutical composition comprises 40 mg/mL to 60 mg/mL of the anti-CD154 antibody or antigen binding fragment thereof, 1 mM to 20 mM glycine, 5% to 10% (w/v) maltose, 0.01% to 0.10% (w/v) polysorbate 80 (PS80), and 10 μg/mL to 30 μg/mL EDTA, at pH 5.0-6.0.

The details of one or more embodiments of the invention are set forth in the description below. Other features and advantages will be apparent from the following detailed description, figures, and the appended claims.

### DETAILED DESCRIPTION OF THE FIGURES

In the Figures:

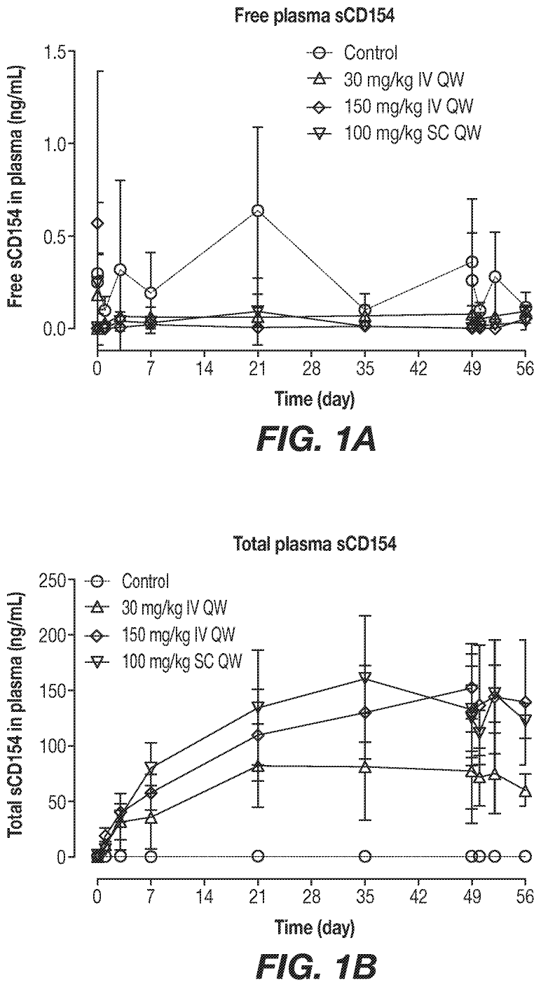

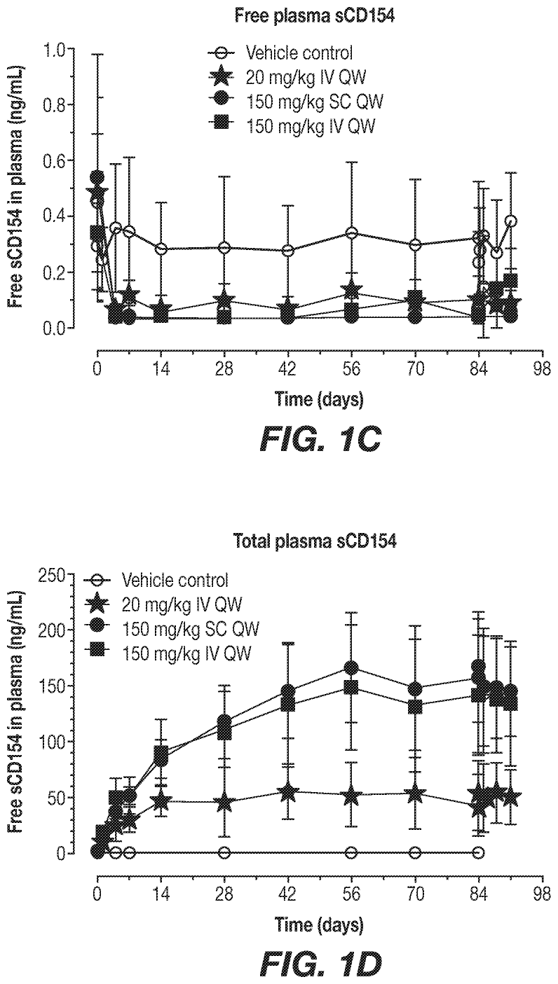

FIGS. 1A-1D show mean plasma total and free soluble CD154 (sCD154) concentrations in rhesus monkeys from the 8-week and 3-month dosing studies with C4LB231 described in Example 1; FIG. 1A and FIG. 1B show the concentration of free sCD154 and total sCD154, respectively, in plasma from the 8-week dosing study for the C4LB231-treated groups; FIG. 1C and FIG. 1D show the concentration of free sCD154 and total sCD154, respectively, in plasma from the 3 month dosing study for the C4LB231-treated groups;

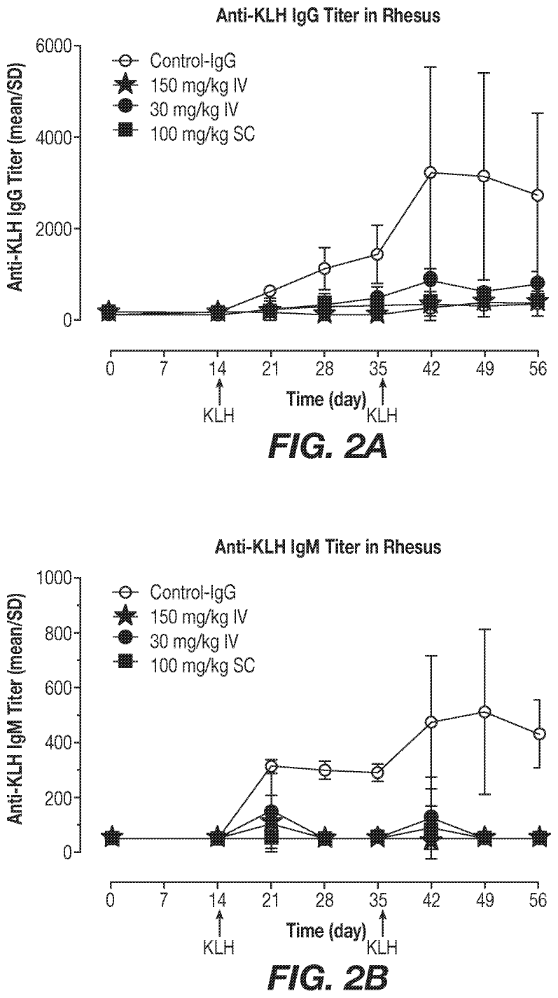

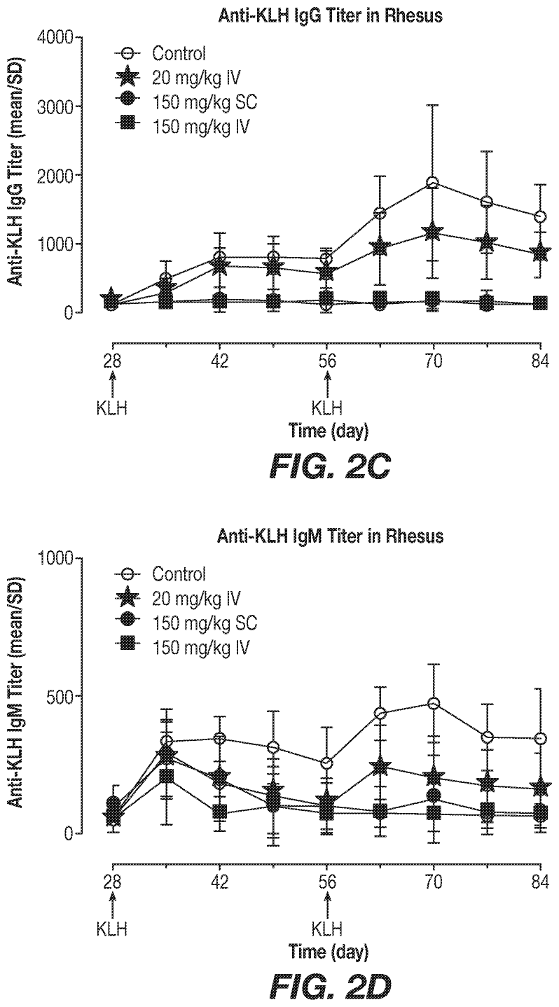

FIGS. 2A-2D show anti-KLH IgG and IgM titers in rhesus monkeys from the 8-week and 3-month dosing studies with C4LB231 described in Example 1; the arrows indicate the timing of KLH antigen immunizations; FIG. 2A and FIG. 2B show the anti-KLH IgG and IgM titers, respectively, from the 8-week dosing study; FIG. 2C and FIG. 2D show the anti-KLH IgG and IgM titers, respectively, from the 3-month dosing study;

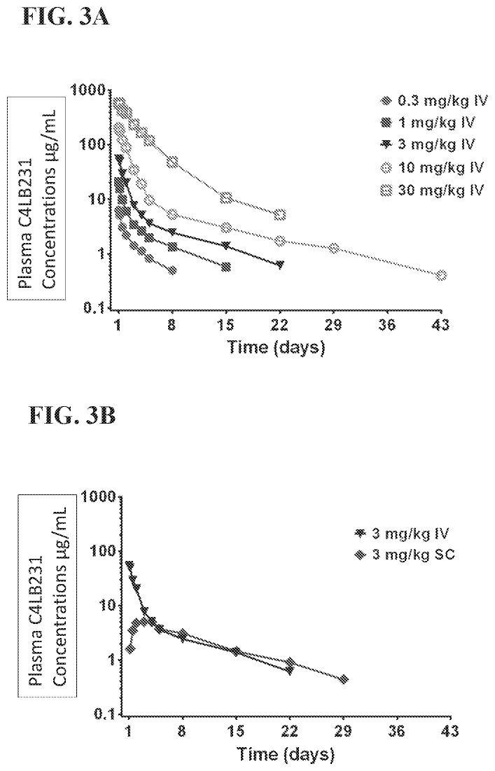

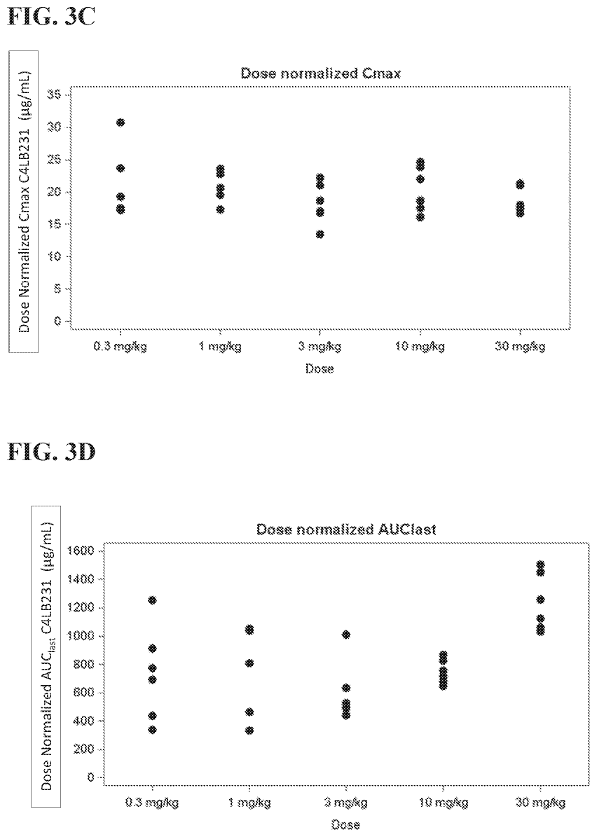

FIGS. 3A-3D show preliminary pharmacokinetics analyses from the clinical study in humans described in Example 2; FIG. 3A shows plasma concentration of C4BL231 over time for each of the cohorts dosed with C4BL231; FIG. 3B shows a comparison of the bioavailability of C4BL231 for intravenous (IV) administration versus subcutaneous (SC) administration at a dose of 3 mg/kg; FIG. 3C shows the dose normalized Cₘₐₓ for individuals in each cohort; FIG. 3D shows the dose normalized AUC for individuals in each cohort;

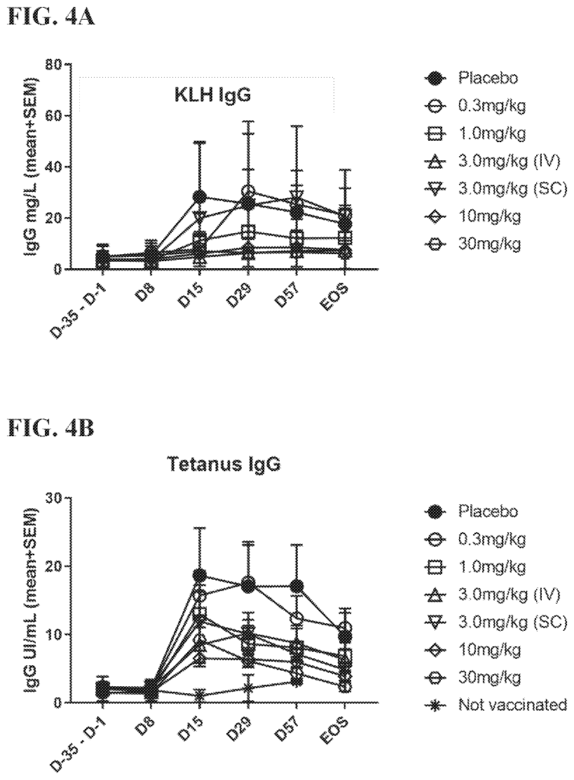

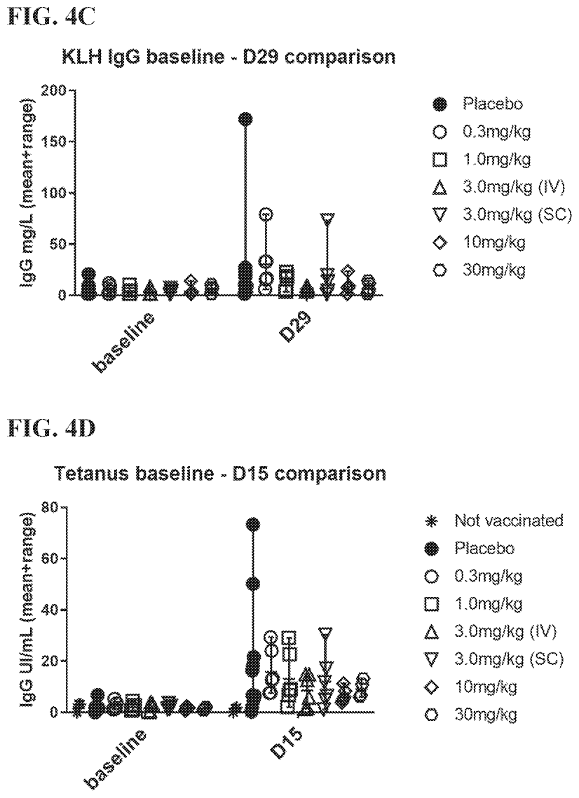

FIGS. 4A-4D show primary anti-KLH IgG response and secondary anti-tetanus toxoid (anti-TT) response from the clinical study in humans described in Example 2; FIG. 4A shows anti-KLH IgG titers; FIG. 4B shows anti-tetanus toxoid IgG titers; FIG. 4C shows a comparison of the anti-KLH IgG concentration at baseline and day 29 (D29) for each cohort; FIG. 4D shows a comparison of the anti-tetanus toxoid IgG concentration at baseline and day 15 (D15) for each cohort;

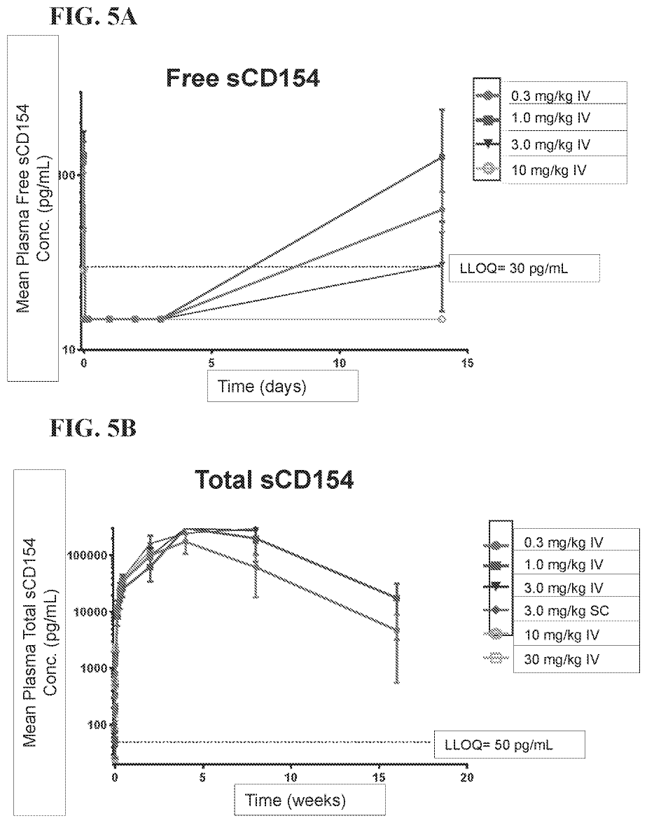

FIGS. 5A-5B show mean plasma free and total soluble CD154 (sCD154) concentrations in humans from the clinical study described in Example 2; FIG. 5A shows free sCD154 concentrations which remained undetectable until 14 days after dosing indicating suppression of the target; FIG. 5B shows total sCD154 concentration which correspondingly increased until reaching a peak value and declined thereafter over time; and

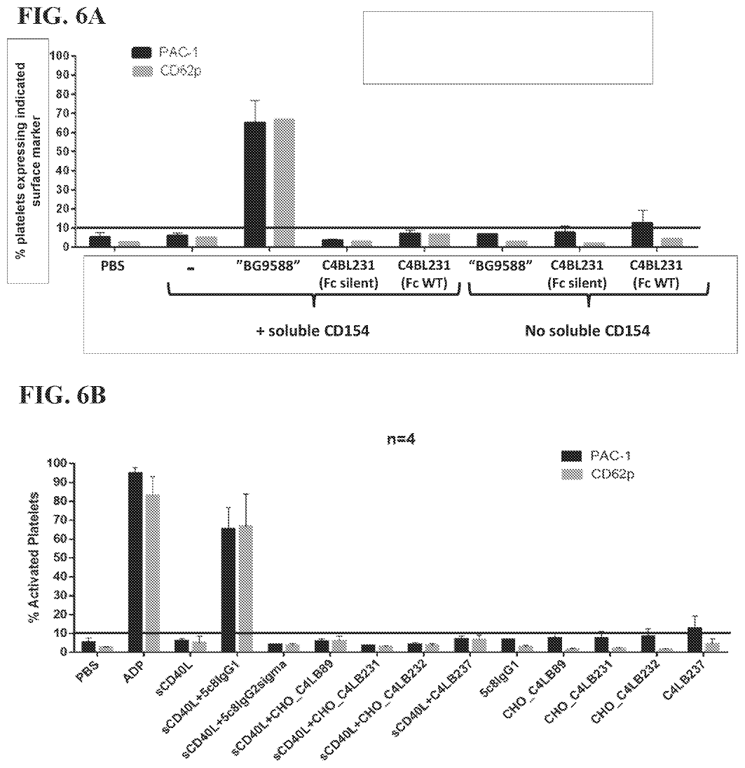

FIGS. 6A-6B show the results of the platelet activation study described in Example 7; FIG. 6A shows platelet activation by C4BL231 having Fc silent and Fc wild-type tail; “Bg9588” (5c8IgG1) is an IgG1 construct similar to the Biogen BG9588 αCD154; “C4BL231 (Fc silent)” refers to C4BL231 IgG1σ antibody; “C4BL231 (Fc WT)” refers to C4LB231 having wild-type IgG1 tail; “+ soluble CD154” refers to incubation of blood with pre-formed complexes of sCD154 and antibody; “− soluble CD154” refers to incubation of blood with antibody alone in the absence of sCD154; FIG. 6B shows platelet activation by other anti-CD154 antibody constructs; data shown is result of N=4 blood donors; ADP was used as positive activation control; sCD40L=sCD154; the following antibodies have the same variable region which is the variable region of C4LB231: C4LB89 (IgG2σ), C4LB231 (IgG1σ), C4LB232 (IgG1σ-YTE), and C4LB237 (IgG1); platelet activation was induced by sCD154-5c8IgG1 complex (but not by sCD154-5c8IgG2σ) confirming Fc-dependent platelet activation; no platelet activation was noted with C4LB231 variable regions on various Fc domains, alone or complexed with sCD154, suggesting additional involvement of the variable region in platelet activation.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

Unless otherwise stated, any numerical value, such as a concentration or a concentration range described herein, are to be understood as being modified in all instances by the term “about.” Thus, a numerical value typically includes ±10% of the recited value. For example, a dosage of 10 mg/kg includes 9 mg/kg to 11 mg/kg. As used herein, the use of a numerical range expressly includes all possible subranges, all individual numerical values within that range, including integers within such ranges and fractions of the values unless the context clearly indicates otherwise.

Throughout this specification and the claims which follow, unless the context requires otherwise, the word “comprise,” and variations such as “comprises” and “comprising,” will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “subject” refers to a mammalian subject, preferably human, diagnosed with or suspected of having an autoimmune disease, whom will be or has been administered an anti-CD154 antibody according to a method of the invention. Diagnosis of an autoimmune disease can be done by a clinician according to clinical diagnostic testing, physical examination of the subject, or any other accepted method for diagnosing a subject with a particular autoimmune disease. As used herein, “a subject suspected of having an autoimmune disease” is a subject that presents signs or symptoms indicative of an autoimmune disease that are discernable to a clinician and/or the subject, but whose suspected diagnosis has not been confirmed by clinical diagnostic testing, physical examination of the subject, or other accepted method for diagnosing a subject with the suspected autoimmune disease.

“CD154” refers to human CD154 (hCD154) (e.g. human CD40L) protein, which belongs to the tumor necrosis factor (TNF) superfamily. Human CD154 full length protein amino acid sequence is shown in SEQ ID NO: 1. Human CD154 is found both on cell membranes as a type II membrane protein and exists in soluble form in plasma. CD154 membrane bound form comprises residues 1-261 of SEQ ID NO: 1, with the transmembrane domain positioned between residues 23-46 and the extracellular domain spanning residues 47-261. The soluble form of human CD154 (shCD154) is formed by proteolytic processing of the membrane bound form, and comprises residues 113-261 of SEQ ID NO: 1 (shCD154 amino acid sequence is shown in SEQ ID NO: 2). Both membrane bound and soluble CD154 form biologically active trimers. “CD154” encompasses the various forms of CD154, including monomer, dimer, trimer, membrane bound and soluble forms as well as naturally occurring variants of human CD154. Soluble human CD154 trimer (shCD154 trimer) is composed of three polypeptide chains each having the amino acid sequence of SEQ ID NO: 2.

CD154 is primarily expressed in a trimeric form on T-cells, particularly on the surface of activated T-cells. However, in addition to its expression on T-cells and in soluble form, CD154 is present in and on platelets. It has been shown that FcγRIIa receptors on platelets play a role in anti-CD154-mediated thromboembolic events (TE). In particular, formation of higher order complexes between anti-CD154 antibodies and shCD154 trimers, e.g., complexes greater than the expected 3:1 ratio of antibody to shCD154 trimer complex, due to anti-CD154 antibody binding to the Fc receptor found on platelets (FcγRIIa) has been shown to contribute to platelet activation, and thus TE events. Platelet activation by anti-CD154 antibodies has been reduced by silencing the Fc domain of the antibody to reduce or eliminate Fc effector function. However, some anti-CD154 antibodies having a silent Fc domain have been shown to mediate platelet activation. Further, some pairs of the variable domains (i.e., VH/VL domain pairs) having CD154 binding specificity were identified which did not mediate platelet activation on either Fc silent or wild-type IgG backbones. This indicates that platelet activation is also epitope dependent, and that both the VH/VL domains (e.g., the epitope the antibody binds to) and higher order complex formation contribute to platelet activation by anti-CD154 antibodies (see, e.g., WO2017/024146). Platelet activation is a well-known process that converts the smooth, nonadherent platelet into a sticky spiculated particle that releases and expresses biologically active substances and acquires the ability to bind the plasma protein fibrinogen. Activation can also occur as a result of the physical stimulus of high fluid shear stress, such as that found at the site of a critical arterial narrowing (Quinn et al., 2005, Platelet Function: assessment, diagnosis, and treatment, Humana Press, pp. 3-20).

As used herein, “an anti-CD154 antibody” refers to a human monoclonal antibody (mAb) of the IgG1σ subtype, or antigen binding fragment thereof, that binds CD154 and blocks the interaction of CD154 with CD40, wherein the antibody or antigen binding fragment thereof comprises heavy chain complementarity determining regions (CDRs) HCDR1, HCDR2, and HCDR3 of SEQ ID NOs: 3, 4, and 5, respectively; and light chain CDRs LCDR1, LCDR2, and LCDR3, of SEQ ID NOs: 6, 7, and 8, respectively. In one embodiment, the anti-CD154 antibody or antigen binding fragment thereof comprises a heavy chain variable region (VH) and a light chain variable region (VL) having the amino acid sequences of SEQ ID NOs: 9 and 10, respectively. Preferably, the anti-CD154 antibody or antigen binding fragment thereof comprises a heavy chain having the amino acid sequence of SEQ ID NO: 11 and a light chain having the amino acid sequence of SEQ ID NO: 12. In preferred embodiments, the IgG1σ backbone of the anti-CD154 antibody or antigen binding fragment thereof includes seven amino acid mutations (L234A, L235A, G237A, P238S, H268A, A330S, and P331S) compared to wild-type IgG1 mAb to silence the Fc domain of the antibody to reduce or eliminate Fc effector function. Thus, in one embodiment, the anti-CD154 antibody or antigen binding fragment thereof does not bind FcγRIIa, FcγRIIb, or FcγRIIIa, and preferably the anti-CD154 antibody or antigen binding fragment thereof does not bind FcγRIIa receptors on platelets. More preferably, the anti-CD154 antibody or antigen binding fragment thereof does not activate human platelets. In some embodiments, reduced platelet activation by an anti-CD154 antibody or antigen binding fragment thereof results from a silenced Fc domain, thus reducing or eliminating Fc effector function and/or the particular VH/VL regions of the antibody, thus reducing or eliminating epitope dependent platelet activation.

In a particular embodiment, the anti-CD154 antibody or antigen binding fragment thereof is C4LB231 as described in International Patent Application Publication WO2017/024146, which is herein incorporated by reference.

Anti-CD154 antibodies can be prepared by any method known in the art in view of the present disclosure for preparing monoclonal antibodies including, but not limited to, hybridoma production. For example, anti-CD154 antibodies can be produced in a mammalian cell line (e.g., Chinese Hamster Ovary (CHO) cell line) using recombinant DNA technology. In particular, C4LB231 and methods of producing C4LB231 are further described in, e.g., International Patent Application Publication WO2017/024146, which is herein incorporated by reference.

The term “safe,” as it relates to a dose, dosage regimen, treatment or method with an anti-CD154 antibody or antigen binding fragment thereof refers to a favorable risk:benefit ratio with an acceptable frequency and/or acceptable severity of treatment-emergent adverse events (referred to as AEs or TEAEs) compared to the standard of care or to another comparator in accordance with the Federal Food, Drug, and Cosmetic Act, as amended (secs. 201-902, 52 Stat. 1040 et seq., as amended; 21 U.S.C. §§ 321-392). In particular, safe as it relates to a dose, dosage regimen, or treatment with an anti-CD154 antibody or antigen binding fragment thereof described herein refers to an acceptable frequency and/or acceptable severity of adverse events associated with administration of the antibody if attribution is considered to be possible, probable, or very likely due to the use of the anti-CD154 antibody or antigen binding fragment thereof. Safety is often measured by toxicity testing to determine the highest tolerable dose or the optimal dose of an active pharmaceutical ingredient needed to achieve the desired benefit. Studies that look at safety also seek to identify any potential adverse effects that may result from exposure to the drug.

As used herein, unless otherwise noted, the term “clinically proven” (used independently or to modify the term “safe”) shall mean that it has been proven by a clinical trial wherein the clinical trial has met the approval standards of U.S. Food and Drug Administration, European Medicines Evaluation Agency (EMEA) or a corresponding national regulatory agency. According to embodiments described herein, the clinical study is a Phase 1, randomized, double-blind, placebo-controlled, single ascending dose study to clinically prove the safety of the drug, i.e., an anti-CD154 antibody or antigen binding fragment, for administration to a subject and for treatment of an autoimmune disease in a subject.

As used herein, the phrases “adverse event,” “treatment-emergent adverse event,” and “adverse reaction” mean any harm, unfavorable, unintended or undesired sign or outcome associated with or caused by administration of a pharmaceutical composition or therapeutic. However, abnormal values or observations are not reported as adverse events unless considered clinically significant by the investigator. As used herein, when referring to an adverse event, “clinically apparent” means clinically significant as determined by a medical doctor or an investigator using standard acceptable to those of ordinary skill in the art. When the harm or undesired outcome of adverse events reaches such a level of severity, a regulatory agency may deem the pharmaceutical composition or therapeutic unacceptable for the proposed use. Examples of unacceptable adverse events or reactions when used in the context of subcutaneous or intravenous administration of an anti-CD154 antibody or antigen binding fragment thereof include, but are not limited to, clinically apparent thromboembolic (TE) events, such as myocardial infarction (MI), pulmonary embolism (PE), and deep vein thrombosis (DVT); severe systemic injection related reactions; increased presence of virus, such as Epstein-Barr virus and/or cytomegalovirus, in blood; and inhibition of immune responses including antibody recall responses (e.g., as measured by antibody recall response to tetanus toxoid), and primary immune responses (e.g., as measured by primary immune response to KLH).

As used herein, “treatment” or “treat” refers to therapeutic treatment. Individuals in need of treatment include those subjects diagnosed with the disorder or a symptom of the disorder. Subjects that may be treated also include those prone to or susceptible to having the disorder, or those in which the disorder is to be prevented. Beneficial or desired clinical results include alleviation of symptoms, diminishment of extent of disease, stabilized (i.e., not worsening) state of disease, delay or slowing of disease progression, amelioration or palliation of the disease state, and remission (whether partial or total), whether detectable or undetectable. Beneficial clinical results include, in a subject who has received treatment, for example reduced proliferation of B cells or dendritic cells, reduction of inflammatory cytokines, adhesion molecules, proteases, immunoglobulins (in instances where the CD40 bearing cell is a B cell), combinations thereof, increased production of anti-inflammatory proteins, a reduction in the number of autoreactive cells, an increase in immune tolerance, inhibition of autoreactive cell survival, and/or a decrease in one or more symptoms mediated by stimulation of CD40-expressing cells by CD154. Clinical response may be assessed using screening techniques such as magnetic resonance imaging (MM) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, chromatography, and the like.

As used herein, a dosage amount of an anti-CD154 antibody or antigen binding fragment thereof in “mg/kg” refers to the amount of the anti-CD154 antibody or antigen binding fragment thereof in milligrams per kilogram of the body weight of a subject to be administered with the anti-CD154 antibody or antigen binding fragment thereof.

In one general aspect, the invention relates to a method of providing clinically proven safe subcutaneous and/or intravenous administration of an anti-CD154 antibody or antigen binding fragment thereof to a subject, preferably a human subject, in need thereof. Preferably, the subject is diagnosed with or suspected of having an autoimmune disease, such as an autoimmune disease that is a systemic autoimmune disease in which T-cells have a role in the initiation and/or progression of the disease. Examples of autoimmune disease with which a subject to be administered an anti-CD154 antibody or antigen binding fragment thereof according to the methods of the invention can be diagnosed with or suspected of having include, but are not limited to, arthritis, rheumatoid arthritis, psoriatic arthritis, ankylosing spondylitis, ulcerative colitis, plaque psoriasis, systemic lupus erythematosus (SLE), Crohn's disease, and Sjögren's Syndrome.

In one preferred embodiment, the autoimmune disease is rheumatoid arthritis. Rheumatoid arthritis is a chronic systemic autoinflammatory disease, causing persisting inflammation and destruction of the synovial lining (synovitis) of joints, particularly in the hands and feet. In addition to the joints, rheumatoid arthritis can eventually affect the skin, heart, lungs, and eyes. T-cells appear to play a role in both the initiation and progression of rheumatoid arthritis, and CD154 also appears to play a role in the pathogenesis of rheumatoid arthritis.

In another preferred embodiment, the autoimmune disease is SLE. SLE targets many organs and tissue, including the skin, blood vessels, muscles, kidneys and lungs. Preclinical and clinical data support a role for CD154 and CD154-expressing T-cells in the initiation and progression of SLE.

In another embodiment, the autoimmune disease is Sjögren's Syndrome. Sjögren's Syndrome is an autoimmune disease in which the immune system attacks the glands that make tears and saliva, causing dry mouth and dry eyes as well as dryness in other parts of the body that need moisture, such as the nose, throat, and skin. Sjögren's Syndrome can also affect other parts of the body, including joints, lungs, kidneys, blood vessels, digestive organs, and nerves.

In another general aspect, the invention relates to a method of providing clinically proven safe treatment of an autoimmune disease in a subject in need thereof, preferably a human subject, the method comprising subcutaneously or intravenously administering to the subject a pharmaceutical composition comprising an anti-CD154 antibody or antigen binding fragment thereof and a pharmaceutically acceptable carrier, wherein a total dosage of the anti-CD154 antibody or antigen binding fragment thereof administered per administration is 0.3 mg/kg to 50 mg/kg. Any of the methods described herein for clinically proven safe administration of the anti-CD154 antibody or antigen binding fragment thereof can be used to provide clinically proven safe treatment of an autoimmune disease.

According to embodiments of the invention, any autoimmune disease can be treated by the methods described herein. Preferably, the autoimmune disease is responsive to treatment with a therapy that targets CD154. Preferably, the autoimmune disease is rheumatoid arthritis, systemic lupus erythematosus (SLE), or Sjögren's Syndrome.

In one embodiment, a method of providing clinically proven safe administration of an anti-CD154 antibody or antigen binding fragment thereof to a subject and/or safe treatment of an autoimmune disease, preferably rheumatoid arthritis, SLE, or Sjögren's Syndrome in a subject, preferably a human subject, comprises subcutaneously or intravenously administering to the subject a pharmaceutical composition comprising an anti-CD154 antibody or antigen binding fragment thereof and a pharmaceutically acceptable carrier, wherein a total dosage of the anti-CD154 antibody or antigen binding fragment thereof administered is 0.3 mg/kg to 50 mg/kg body weight of the subject per administration.

In one embodiment, the pharmaceutical composition is administered subcutaneously. Subcutaneous administration refers to administration under the skin, in which a drug or therapeutic is injected into the tissue layer between the skin and muscle. Medication administered via subcutaneous administration is usually absorbed more slowly than if injected into a vein. When administration of an anti-CD154 antibody or antigen binding fragment thereof is via subcutaneous injection, the total dosage of the anti-CD154 antibody or antigen binding fragment thereof to be administered to the subject per administration can be administered in a single subcutaneous injection, or in multiple subcutaneous injections, such as 1, 2, 3, 4, 5, or more subcutaneous injections.

In another embodiment, the pharmaceutical composition is administered intravenously. Intravenous administration refers to administration directly into a vein. Intravenous administration can be via injection (e.g., with a syringe at higher pressures) or via infusion (e.g., using the pressure supplied by gravity). Intravenous administration is typically the quickest method for delivering a drug or therapeutic throughout the body, because the drug or therapeutic is carried by circulation. When administration of an anti-CD154 antibody or antigen binding fragment thereof is via intravenous administration, administration can be by intravenous infusion or injection, and is preferably via infusion. For example, the total dosage of an anti-CD154 antibody or antigen binding fragment thereof to be administered to the subject per administration can be administered by intravenous infusion over a period of about 30 minutes to 180 minutes, preferably 60 minutes to 120 minutes, such as 30 minutes, 60 minutes, 90 minutes, 120 minutes, 150 minutes, or 180 minutes.

The total dosage of an anti-CD154 antibody or antigen binding fragment thereof per administration is selected so as to provide safe administration and/or safe treatment by subcutaneous or intravenous administration as determined in clinical trials. According to embodiments of the invention, a total dosage of the anti-CD154 antibody or antigen binding fragment thereof administered per administration is, for example, 0.3 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg, or 50 mg/kg, or any dosage in between. The total dosage of the anti-CD154 antibody or antigen binding fragment thereof can be administered once per day, once per week, once every two weeks, once every three weeks, once per month, once every six months, etc. for a period of one day, one week, one month, six months, 1 year, 2 years or longer. For example, a total dosage of 0.3 mg/kg to 50 mg/kg of the anti-CD154 antibody or antigen binding fragment thereof can be administered per administration (e.g., once per day for at least one day) by a single subcutaneous injection, or multiple subcutaneous injections (e.g., 2 to 5 injections) at substantially the same time, i.e., over a time period of 0 minutes to 1 hour, such as 5 minutes, 10 minutes, 15 minutes, 20 minutes, 25 minutes, 30 minutes, 35 minutes, 40 minutes, 45 minutes, 50 minutes, 55 minutes, or 1 hour. Alternatively, a total dosage of 0.3 mg/kg to 50 mg/kg of the anti-CD154 antibody or antigen binding fragment thereof can be administered per administration (e.g., once per day for at least one day) by intravenous infusion over a time period of about 30 minutes to 3 hours, preferably 60 minutes to 120 minutes. Multiple administrations of the anti-CD154 antibody or antigen binding fragment thereof, each at a total dosage of 0.3 mg/kg to 50 mg/kg, can be administered to a subject in need thereof.

Pharmaceutical compositions suitable for use in the methods of the invention are formulated for subcutaneous administration or intravenous administration. Examples of formulations suitable for subcutaneous and/or intravenous administration include, but are not limited to, solutions, suspensions, emulsions, and dry products that can be dissolved or suspended in a pharmaceutically acceptable carrier for injection or infusion. In a preferred embodiment, a pharmaceutical composition comprising an anti-CD154 antibody or antigen binding fragment thereof for use in the methods of the invention is formulated as a solution.

A concentration of an anti-CD154 antibody or antigen binding fragment thereof included in pharmaceutical compositions used in the invention can vary. Typically, the concentration of the anti-CD154 antibody or antigen binding fragment thereof is 1 mg/mL to 100 mg/mL, such as 1 mg/mL, 10 mg/mL, 20 mg/mL, 30 mg/mL, 40 mg/mL, 50 mg/mL, 60 mg/mL, 70 mg/mL, 80 mg/mL, 90 mg/mL, or 100 mg/mL, or any concentration in between. Preferably, the concentration of the anti-CD154 antibody or antigen binding fragment thereof is 40 mg/mL to 60 mg/mL, for instance 50 mg/mL. Pharmaceutical compositions comprising an anti-CD154 antibody or antigen binding fragment thereof at such concentrations can be administered to a subject directly, or such compositions can be diluted with a suitable diluent (e.g., sterile 1% to 10% glucose solution, for instance 5% glucose solution) to an appropriate volume for administration, particularly when the composition is to be administered via intravenous infusion. For example, pharmaceutical compositions containing an anti-CD154 antibody or antigen binding fragment thereof at a concentration of 40 mg/mL to 60 mg/mL can be diluted in diluent to a total volume of 200 mL to 300 mL, for instance 250 mL to be administered by intravenous infusion over a period of about 60 minutes to 120 minutes.

Pharmaceutical compositions for use in the invention further comprise one or more pharmaceutically acceptable carriers, such as those widely employed in the art of drug manufacturing, and particularly antibody drug manufacturing. As used herein, the term “carrier” refers to any excipient, diluent, buffer, stabilizer, or other material well known in the art for pharmaceutical formulations. Pharmaceutically acceptable carriers in particular are non-toxic and should not interfere with the efficacy of the active ingredient. The pharmaceutically acceptable carriers include excipients and/or additives suitable for use in the pharmaceutical compositions known in the art, e.g., as listed in “Remington: The Science & Practice of Pharmacy”, 19th ed., Williams & Williams, (1995), and in the “Physician's Desk Reference”, 52nd ed., Medical Economics, Montvale, N.J. (1998), the disclosures of which are entirely incorporated herein by reference.

According to embodiments of the invention, a pharmaceutical composition for use in the invention comprises an anti-CD154 antibody or antigen binding fragment thereof and a pharmaceutically acceptable carrier. In some embodiments, the pharmaceutically acceptable carrier comprises one or more amino acids, such as arginine, glycine, histidine and/or methionine, one or more carbohydrates, such as lactose, maltose, sucrose, one or more surfactants, such as polysorbate 20, polysorbate 80, and one or more chelators, such as ethylenediaminetetracetic acid (EDTA), and ethylenediamine-N,N′-disuccinic acid (EDDS). Preferably, the pharmaceutical composition has a pH of 5 to 6, such as a pH of 5.0, 5.1, 5.2, 5.3, 5.4, 5.5, 5.6, 5.7, 5.8, 5.9, or 6.0, or any value in between.

In some embodiments, a pharmaceutical composition for use in the invention comprises histidine, arginine, and/or glycine at a concentration of 1 mM to 50 mM, 5 mM to 50 mM, 5 mM to 30 mM, 5 mM to 20 mM, 5 mM to 15 mM or 5 mM to 10 mM. For example, the concentration of histidine, arginine, and/or glycine can be 1 mM, 2 mM 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM or 50 mM, or any concentration in between.

In some embodiments, a pharmaceutical composition for use in the invention comprises sucrose, lactose, and/or maltose at a concentration of 1% to 10% weight by volume (w/v), 5% to 10% (w/v), or 7% to 9% (w/v). For example, the concentration of sucrose, lactose, and/or maltose can be 1% (w/v), 1.5% (w/v), 2% (w/v), 2.5% (w/v), 3% (w/v), 3.5% (w/v), 4% (w/v), 4.5% (w/v), 5% (w/v), 5.5% (w/v), 6% (w/v), 6.5% (w/v), 7% (w/v), 7.5% (w/v), 8% (w/v), 8.5% (w/v), 9% (w/v), 9.5% (w/v), or 10% (w/v), or any concentration in between.

In some embodiments, a pharmaceutical composition for use in the invention comprises polysorbate 20 (PS20) and/or polysorbate 80 (PS80) at a concentration of 0.01% (w/v) to 0.1% (w/v), 0.01% (w/v) to 0.08% (w/v), or 0.02% (w/v) to 0.05% (w/v). For example, the concentration of polysorbate 20 and/or polysorbate 80 can be 0.01%, 0.02%, 0.03%, 0.04%, 0.05%, 0.06%, 0.07%, 0.08%, 0.09% or 0.1% (w/v), or any concentration in between.

In some embodiments, a pharmaceutical composition for use in the invention comprises ethylenediaminetetracetic acid (EDTA) and/or ethylenediamine-N,N′-disuccinic acid (EDDS) at a concentration of 1 μg/mL to 50 μg/mL, 1 μg/mL to 30 μg/mL or 10 μg/mL to 30 μg/mL. For example, the concentration of EDTA and/or EDDS can be 1 μg/mL, 5 μg/mL, 10 μg/mL, 15 μg/mL, 20 μg/mL, 25 μg/mL, 30 μg/mL, 35 μg/mL, 40 μg/mL, 40 μg/mL, 45 μg/mL, or 50 μg/mL, or any concentration in between.

Pharmaceutical compositions comprising an anti-CD154 antibody or antigen binding fragment thereof for use in the invention can be prepared by any method known in the art in view of the present disclosure. For example, an anti-CD154 antibody or antigen binding fragment thereof can be mixed with one or more pharmaceutically acceptable carriers to obtain a solution. The solution can be stored as a frozen liquid at a controlled temperature ranging from −40° C.±10° C. to −70° C.±20 ° C. and under protection from light exposure in an appropriate vial until administered to the subject.

According to embodiments of the invention, a variety of factors can be analyzed to determine by clinical trials such as those described herein whether a particular dosage of the anti-CD154 antibody or antigen binding fragment thereof provides for safe subcutaneous and/or intravenous administration. For example, safety of a certain dosage of subcutaneously and/or intravenously administered anti-CD154 antibody or antigen binding fragment thereof can be assessed by immunogenicity studies (e.g., measuring the production of antibodies to the anti-CD154 antibody, e.g., anti-C4LB231 antibodies); determining the effects on blood biomarkers, such as serum proteins (e.g., cytokines, chemokines, and inflammatory proteins) by protein profiling; measuring viral reaction (e.g., viral reactivation or viral load of Epstein-Barr virus and/or cytomegalovirus); determining a level of platelet activation; primary and recall antigen challenge studies; and measuring total and free plasma levels of soluble CD154 (sCD154). The safety of subcutaneously and/or intravenously administered anti-CD154 antibody or antigen binding fragment thereof can also be monitored by physical examination of the subject; observation of local injection site reactions, systemic injection related reactions, and other allergic reactions; electrocardiograms; clinical laboratory tests; vital signs; and monitoring of other adverse events, such as thromboembolic events.

In some embodiments, clinically proven safe administration of an anti-CD154 antibody or antigen binding fragment thereof and/or clinically proven safe treatment of an autoimmune disease is determined by measuring platelet activation. Activation of platelets results in activation of intracellular signaling pathways resulting in upregulation of platelet surface expression of P-selectin and increased binding affinity of fibrinogen to integrin receptors αIIbβ3. Platelet activation can therefore be measured by measuring increased P-selectin surface expression or binding of probe ligand e.g. PAC-1 to αIIbβ3 integrin on platelets using for example flow cytometry. Platelet activation can thus be measured and/or quantified by flow cytometry of cells expressing clotting markers (e.g., P-selectin), or by other methods known in the art in view of the present disclosure for counting platelets. In one embodiment, platelet activation is determined by measuring P-selectin surface expression on platelets. In another embodiment, platelet activation is determined by measuring binding of probe ligand to αIIbβ3 integrin on platelets.

In some embodiments, clinically proven safe administration of an anti-CD154 antibody or antigen binding fragment thereof and/or clinically proven safe treatment of an autoimmune disease is determined by measuring primary and/or recall immune responses to antigens. Primary and recall (or memory) T-cell-dependent responses can be used to evaluate the efficacy and safety of a therapy that is an immune modulating agent. Primary and recall T-cell dependent responses can be measured by administering an antigen to a subject, preferably after administration of the anti-CD154 antibody or antigen binding fragment thereof, to evaluate the effect of administration of the anti-CD154 antibody or antigen binding fragment thereof on such immune responses. For example, an antigen can be administered one day, two days, three days, four days, five days, six days, seven days, eight days, nine days, ten days, one month, six months, or one year or longer after the anti-CD154 antibody or antigen binding fragment thereof is administered to the subject. Preferably, the antigen is administered one to ten days, more preferably two to four days, for instance three days, after the anti-CD154 antibody or antigen binding fragment thereof is administered. The antigen can be administered by any method known in the art in view of the present disclosure, e.g., by intramuscular or subcutaneous injection. The antigen is preferably administered to the subject at an anatomical location that is distinct from the site of administration of the anti-CD154 antibody or antigen binding fragment thereof.

Antigens that can be administered to a subject for the purpose of evaluating primary immune response include antigens to which humans rarely demonstrate evidence of immunologic exposure (e.g., keyhole limpet hemocyanin). Antigens that can be administered to a subject for the purpose of evaluating recall immune response include antigens to which most humans have been previously exposed, for example through vaccination (e.g., tetanus toxoid).

In one embodiment, primary immune response to keyhole limpet hemocyanin (KLH) is measured. KLH is an antigen which is used to assess T-cell-dependent immune response. KLH is derived from the inedible mollusk, Megathura crenulate. Humans rarely demonstrate evidence of immunologic exposure to KLH, for example anti-immunoglobulin G (IgG) or IgM antibodies to KLH. Therefore, KLH challenges can be used to assess the effects of immune modulators on specific primary antigen responses.

In one embodiment, recall (or memory) immune response to tetanus toxoid (TT) is measured. Tetanus toxoid vaccine, which comprises peptide antigens, is commonly administered for vaccination during childhood or early adolescence, often with boosters in later adolescence and adulthood. Thus, antibody responses to challenges with tetanus toxoid are often considered a recall, or memory, T-cell dependent response. Such recall responses typically have a higher threshold for modulation and evaluating the effect of immune modulating agents on recall responses can provide insight into the safety of the agent. Primary and recall immune responses to an administered antigen can be measured by any method known in the art in view of the present disclosure for assessing cellular and/or humoral mediated immune responses. Measurement of cellular immunity can be performed by measurement of cytokine profiles secreted by activated effector cells (e.g., by enzyme-linked immunospot (ELISpot) assay), by determining the activation status of immune effector cells (e.g., T-cell proliferation assays), and/or by assaying for antigen-specific T lymphocytes in a sensitized subject (e.g., peptide-specific lysis in a cytotoxicity assay). The ability to stimulate a humoral response can be determined by antibody binding and/or competition in binding. For example, titers of antibodies produced in response to administration of an antigen can be measured by enzyme-linked immunosorbent assay (ELISA). ELISpot can also be used to assess humoral immune response to identify and enumerate the number of cells secreting an antibody produced in response to administration of an antigen.

In some embodiments of the invention, antibodies produced in response to KLH and/or tetanus toxoid administration are assessed to evaluate the effect of administration of an anti-CD154 antibody or antigen binding fragment thereof on primary and/or recall immune response. For example, serum samples can be collected from subjects subsequent to administration of an antigen (e.g., KLH and/or tetanus toxoid). The serum samples can be analyzed for antibodies to the administered antigens by, for example, ELISA. In one embodiment, antibodies to KLH (e.g., anti-KLH IgG and IgM antibodies) are detected. In another embodiment, antibodies to tetanus toxoid (e.g., anti-tetanus IgG antibodies) are detected.

According to embodiments of the invention, a potentially clinically significant inhibition of antibody recall responses includes, e.g., failure to induce at least a 2-fold increase of anti-tetanus IgG antibody levels compared to pre-tetanus toxoid challenge in individuals who have pre-existing protective antibody levels. In other embodiments, a potentially clinical significant inhibition of primary immune response includes, e.g., failure to induce any detected level of anti-KLH antibodies.

In some embodiments, clinically proven safe administration of an anti-CD154 antibody or antigen binding fragment thereof and/or clinically proven safe treatment of an autoimmune disease is determined by viremia, such as by measuring viral load or viremia. Viremia (i.e., the presence of viruses in the blood) that is below a clinically significant level is indicative of safe administration and/or safe treatment. Viremia, or viral load assessment can be determined by measuring the number of viral DNA copies of a virus in a blood sample. A “clinically significant level” as used herein with respect to viremia or viral load means viral DNA copies of ≥10,000 copies/mL blood. According to embodiments of the invention, clinically proven safe administration of an anti-CD154 antibody or antigen binding fragment thereof and/or clinically proven safe treatment of an autoimmune disease comprises viral load assessment or viremia below a clinically significant level, i.e., <10,000 viral DNA copies/mL of sample from the subject. The sample can be blood, serum, or plasma. Viral load or viremia can be measured by any method known in the art in view of the present disclosure including, but not limited to polymerase chain reaction (PCR) or quantitative real-time PCT (qRT-PCR) using primers specific to viral DNA. See, e.g., Rosenzweig et al., Development of a quantitative assay to measure EBV viral load in patients with autoimmune type 1 diabetes and healthy subjects, J of Virological Methods. 2010; 164:111-115; Verkruyse et al., Once daily ganciclovir as initial pre-emptive therapy delayed until threshold CMV load X10000 copies/ml: a safe and effective strategy for allogeneic stem cell transplant patients. Bone Marrow Transplantation. 2006; 37:51-56, the content of which are incorporated herein by reference. Viral load or viremia can be determined for viruses including, but not limited to, Epstein-Barr virus (EBV) and cytomegalovirus (CMV).

In some embodiments, clinically proven safe administration of an anti-CD154 antibody or antigen binding fragment thereof and/or clinically proven safe treatment of an autoimmune disease is determined based on the occurrence of any thromboembolic event in the subject. Thromboembolic events include, but are not limited to, myocardial infarction (MI), pulmonary embolism (PE), and deep vein thrombosis (DVT). The occurrence of a thromboembolic event can also be determined based on changes in blood coagulation factors suggestive of thrombosis and/or coagulopathy. Such changes in blood coagulation factors that are suggestive or indicative of thromboembolic events include, but are not limited to, decreases in platelet counts, hemoglobin, haptoglobin, or fibrinogen levels; increases in D-dimer values, prothrombin time (PT), partial thromboblastin time (PTT) or international normalized ration (INR); and morphologic changes in red blood cells (RBCs) that are consistent with thrombosis or embolism.

In some embodiments, clinically proven safe administration of an anti-CD154 antibody or antigen binding fragment thereof and/or clinically proven safe treatment of an autoimmune disease is determined by measuring an amount of soluble CD154 (sCD154) in a sample obtained from a subject. The amount of sCD154 can be measured by any method known in the art in view of the present disclosure, e.g., ELISA, electrochemiluminescence immunoassay (ECLIA), etc.

In one exemplary regimen of providing clinically proven safe subcutaneous administration of an anti-CD154 antibody or antigen binding fragment thereof and/or clinically proven safe treatment of an autoimmune disease with an anti-CD154 antibody or antigen binding fragment thereof, a subject is subcutaneously administered a pharmaceutical composition comprising 50 mg/mL of the anti-CD154 antibody or antigen binding fragment thereof. The total volume of the composition administered is appropriately adjusted to provide the target dosage, i.e., 0.3 mg/kg to 50 mg/kg body weight of the subject of the anti-CD154 antibody or antigen binding fragment thereof, in a single subcutaneous injection, in multiple subcutaneous injections, or by intravenous infusion.

### Embodiments

Embodiment 1 is a method of providing clinically proven safe administration of an anti-CD154 antibody or antigen binding fragment thereof to a subject in need thereof, the method comprising subcutaneously or intravenously administering to the subject a pharmaceutical composition comprising the anti-CD154 antibody or antigen binding fragment thereof and a pharmaceutically acceptable carrier, wherein a total dosage of the anti-CD154 antibody or antigen binding fragment thereof administered is 0.3 mg/kg to 50 mg/kg body weight of the subject per administration.

Embodiment 1a is the method of embodiment 1, wherein the anti-CD154 antibody or antigen binding fragment thereof comprises heavy chain complementarity determining regions (CDRs) HCDR1, HCDR2, and HCDR3 of SEQ ID NOs: 3, 4, and 5, respectively, and light chain CDRs LCDR1, LCDR2, and LCDR3, of SEQ ID NOs: 6, 7, and 8, respectively.

Embodiment 1b is the method of embodiment 1, wherein the anti-CD154 antibody or antigen binding fragment thereof comprises a heavy chain variable region (VH) and a light chain variable region (VL) having amino acid sequences of SEQ ID NOs: 9 and 10, respectively.

Embodiment 1c is the method of embodiment 1, wherein the anti-CD154 antibody or antigen binding fragment thereof comprises an IgG1σ backbone having the amino acid mutations L234A, L235A, G237A, P238S, H268A, A330S, and P331S compared to a wild-type IgG backbone.

Embodiment 1d is the method of embodiment 1, wherein the anti-CD154 antibody or antigen binding fragment thereof comprises a heavy chain having the amino acid sequence of SEQ ID NO: 11 and a light chain having the amino acid sequence of SEQ ID NO: 12.

Embodiment 2 is a method of providing clinically proven safe treatment of an autoimmune disease in a human subject in need thereof, the method comprising subcutaneously or intravenously administering to the subject a pharmaceutical composition comprising an anti-CD154 antibody or antigen binding fragment thereof and a pharmaceutically acceptable carrier, wherein a total dosage of the anti-CD154 antibody or antigen binding fragment thereof administered is 0.3 mg/kg to 50 mg/kg body weight of the subject per administration.

Embodiment 2a is the method of embodiment 2, wherein the anti-CD154 antibody or antigen binding fragment thereof comprises heavy chain complementarity determining regions (CDRs) HCDR1, HCDR2, and HCDR3 of SEQ ID NOs: 3, 4, and 5, respectively, and light chain CDRs LCDR1, LCDR2, and LCDR3, of SEQ ID NOs: 6, 7, and 8, respectively.

Embodiment 2b is the method of embodiment 2, wherein the anti-CD154 antibody or antigen binding fragment thereof comprises a heavy chain variable region (VH) and a light chain variable region (VL) having amino acid sequences of SEQ ID NOs: 9 and 10, respectively.

Embodiment 2c is the method of embodiment 2, wherein the anti-CD154 antibody or antigen binding fragment thereof comprises an IgG1σ backbone having the amino acid mutations L234A, L235A, G237A, P238S, H268A, A330S, and P331S compared to a wild-type IgG backbone.

Embodiment 2d is the method of embodiment 1, wherein the anti-CD154 antibody or antigen binding fragment thereof comprises a heavy chain having the amino acid sequence of SEQ ID NO: 11 and a light chain comprising the amino acid sequence of SEQ ID NO: 12.

Embodiment 3 is the method of any one of embodiments 2-2d, wherein the autoimmune disease is a systemic autoimmune disease in which T-cells have a role in the initiation and/or progression of the disease.

Embodiment 4 is the method of any one of embodiments 2-2d, wherein the autoimmune disease is selected from the group consisting of arthritis, rheumatoid arthritis, psoriatic arthritis, ankylosing spondylitis, ulcerative colitis, plaque psoriasis, systemic lupus erythematosus (SLE), Crohn's disease, and Sjögren's Syndrome.

Embodiment 5 is the method of any one of embodiments 2-2d, wherein the autoimmune disease is rheumatoid arthritis.

Embodiment 6 is the method of any one of embodiments 2-2d, wherein the autoimmune disease is systemic lupus erythematosus (SLE).

Embodiment 7 is the method of any one of embodiments 1 to 6, wherein the total dosage of the anti-CD154 antibody or antigen binding fragment thereof administered is 0.3 mg/kg, 1 mg/kg, 3 mg/kg, 10 mg/kg, 30 mg/kg or 50 mg/kg or any dosage in between.

Embodiment 8 is the method of any one of embodiments 1 to 7, wherein the pharmaceutical composition is administered subcutaneously.

Embodiment 9 is the method of embodiment 8, wherein the total dosage of the anti-CD154 antibody or antigen binding fragment thereof is administered in one, two, three, or four subcutaneous injections per administration.

Embodiment 10 is the method of any one of embodiments 1 to 7, wherein the pharmaceutical composition is administered intravenously.

Embodiment 11 is the method of any one of embodiments 1 to 10, wherein the clinically proven safe administration of the anti-CD154 antibody or antigen binding fragment thereof and/or clinically proven safe treatment of the autoimmune disease comprises viremia of <10,000 copies of viral DNA of at least one virus per mL of sample from the subject, preferably per mL of blood, serum, or plasma from the subject.

Embodiment 12 is the method of embodiment 11, wherein the virus is at least one selected from the group consisting of Epstein-Barr virus (EBV) and cytomegalovirus (CMV).

Embodiment 13 is the method of embodiment 11 or 12, wherein the viral DNA is determined by PCR.

Embodiment 14 is the method of any one of embodiments 1 to 13, wherein the clinically proven safe administration of the anti-CD154 antibody or antigen binding fragment thereof and/or clinically proven safe treatment of the autoimmune disease comprises an immune response comprising a recall response, such as a recall response to tetanus toxoid.

Embodiment 15 is the method of embodiment 14, wherein the recall response is measured by detecting anti-tetanus antibodies in the subject administered with a booster immunization of the tetanus toxoid.

Embodiment 15a is the method of embodiment 15, further comprising administering to the subject a booster immunization of the tetanus toxoid after the administration of the anti-CD154 antibody or antigen binding fragment thereof, and detecting anti-tetanus antibodies in serum of the subject to thereby determine the recall response to tetanus toxoid.

Embodiment 16 is the method of any one of embodiments 1 to 15, wherein the clinically proven safe administration of the anti-CD154 antibody or antigen binding fragment thereof and/or clinically proven safe treatment of the autoimmune disease comprises an immune response comprising a primary response, such as a primary response to KLH.

Embodiment 17 is the method of embodiment 16, wherein the primary response is measured by detecting anti-KLH antibodies in the subject administered with a primary immunization of KLH.

Embodiment 17a is the method of embodiment 17, further comprising administering to the subject a primary immunization of the KLH after the administration of the anti-CD154 antibody or antigen binding fragment thereof, and detecting anti-KLH antibodies in serum of the subject to thereby determine the primary response to KLH.

Embodiment 18 is the method of any one of embodiments 1 to 17a, wherein the clinically proven safe administration of the anti-CD154 antibody or antigen binding fragment thereof and/or clinically proven safe treatment of the autoimmune disease does not result in any clinically apparent thromboembolic (TE) event in the subject.

Embodiment 19 is the method of embodiment 18, wherein the TE event comprises at least one of myocardial infarction (MI), pulmonary embolism (PE), and deep vein thrombosis (DVT).

Embodiment 20 is the method of embodiment 18, wherein occurrence of a TE event is determined based on changes in blood coagulation factors.

Embodiment 21 is the method of any one of embodiments 1 to 20, wherein the administration of the anti-CD154 antibody or antigen binding fragment thereof does not result in activation of platelets.

Embodiment 22 is the method of embodiment 21, wherein platelet activation is determined by measuring P-selectin surface expression on platelets.

Embodiment 23 is the method of embodiment 21, wherein platelet activation is determined by measuring binding of probe ligand to αIIbβ3 integrin on platelets.

Embodiment 24 is the method of any one of embodiments 1 to 23, wherein the pharmaceutical composition comprises the anti-CD154 antibody or antigen binding fragment thereof at a concentration of 1 mg/mL to 100 mg/mL.

Embodiment 25 is the method of embodiment 24, wherein the concentration of the anti-CD154 antibody or antigen binding fragment thereof is 1 mg/mL, 10 mg/mL, 20 mg/mL, 30 mg/mL, 40 mg/mL, 50 mg/mL, 60 mg/mL, 70 mg/mL, 80 mg/mL, 90 mg/mL, or 100 mg/mL, or any concentration in between.

Embodiment 26 is the method of any one of embodiments 1 to 25, wherein the pharmaceutical comprises 40 mg/mL to 60 mg/mL of the anti-CD154 antibody or antigen binding fragment thereof, 1 mM to 20 mM arginine, 5% to 10% (w/v) lactose, 0.01% to 0.10% (w/v) polysorbate 80 (PS80), and 10 μg/mL to 30 μg/mL EDDS, at pH 5.0-6.0.

Embodiment 26a is the method of embodiment 26, wherein the pharmaceutical composition comprises 50 mg/mL of the anti-CD154 antibody or antigen binding fragment thereof, 10 mM arginine, 8.5% (w/v) lactose, 0.04% (w/v) polysorbate 80 (PS80), and 20 μg/mL EDDS, at pH 5.6.

Embodiment 27 is the method of any one of embodiments 1 to 25, wherein the pharmaceutical comprises 40 mg/mL to 60 mg/mL of the anti-CD154 antibody or antigen binding fragment thereof, 1 mM to 20 mM histidine, 5% to 10% (w/v) sucrose, 0.01% to 0.10% (w/v) polysorbate 20 (PS20), and 10 μg/mL to 30 μg/mL EDTA, at pH 5.0-6.0.

Embodiment 27a is the method of embodiment 27, wherein the pharmaceutical composition comprises 50 mg/mL of the anti-CD154 antibody or antigen binding fragment thereof, 10 mM histidine, 8.5% (w/v) sucrose, 0.04% (w/v) polysorbate 20 (PS20), and 20 μg/mL ethylenediaminetetracetic acid (EDTA), at pH 5.6.

Embodiment 28 is the method of any one of embodiments 1 to 25, wherein the pharmaceutical comprises 40 mg/mL to 60 mg/mL of the anti-CD154 antibody or antigen binding fragment thereof, 1 mM to 20 mM glycine, 5% to 10% (w/v) maltose, 0.01% to 0.10% (w/v) polysorbate 80 (PS80), and 10 μg/mL to 30 μg/mL EDTA, at pH 5.0-6.0.

Embodiment 28a is the method of embodiment 28, wherein the pharmaceutical composition comprises 50 mg/mL of the anti-CD154 antibody or antigen binding fragment thereof, 10 mM glycine, 8.5% (w/v) maltose, 0.04% (w/v) polysorbate 80 (PS80), and 20 μg/mL ethylenediaminetetracetic acid (EDTA), at pH 5.6.

Embodiment 29 is the method of any one of embodiments 1 to 28, wherein the subject is a human.

Embodiment 30 is the method of any one of embodiments 1 to 29, wherein the subject is diagnosed with or suspected of having an autoimmune disease.

The following examples of the invention are to further illustrate the nature of the invention. It should be understood that the following examples do not limit the invention and the scope of the invention is to be determined by the appended claims.

### EXAMPLES

### Example 1

### In Vivo Repeat Dose Studies in Rhesus Monkeys to Evaluate Toxicity of Parenterally Administered Anti-CD154 Antibody

The tolerability and potential thromboembolism risk of intravenously (IV) or subcutaneously (SC) administered anti-CD154 antibody in rhesus monkeys were evaluated. Rhesus monkeys were selected as the more sensitive species in non-clinical safety evaluation of the target anti-CD154 antibody over cynomolgus monkeys although both are pharmacologically relevant, because administration of an anti-CD154 IgG1 antibody known to induce thromboembolism in humans led to the development of pulmonary thromboembolism in rhesus monkeys, but not in naïve cynomolgus monkeys.

More specifically, in a pharmacokinetic (PK)/pharmacodynamic (PD) study in cynomolgus monkeys, twenty-two monkeys were administered a single dose of C4LB231 ranging from 0.05 mg/kg to 5 mg/kg by IV administration, or a single dose of 5 mg/kg C4LB231 by subcutaneous administration. Dose-proportional increases in peak serum concentrations (Cₘₐₓ) of C4LB231 were observed. However, Cₘₐₓ serum concentrations of C4LB231 decreased rapidly possibly due to high incidence of anti-drug antibodies (ADA). Of the twenty-two monkeys treated with C4LB231, ten monkeys dosed with C4LB231 received a single dose of KLH challenge on day 2. PK of C4LB231 in these KLH-challenged animals was comparable to animals receiving the same dose of C4LB231 without KLH challenge.

Thus, although cynomolgus monkeys are also pharmacologically relevant (PK/PD), rhesus monkeys is considered the more sensitive species for toxicity studies of the target anti-CD154 antibody to evaluate potential risk of thromboembolism.

I. 8-Week Dose Study

Rhesus monkeys were administered the anti-CD154 antibody C4LB231 intravenously (IV) or subcutaneously (SC) once weekly for 8 weeks. The experimental design of the study is shown in Table 1 below. The anti-CD154 IgG1 antibody 5c8 (Biogen), which is known in induce thromboembolism based on observations during previous clinical studies, was used as the positive control. The control vehicle was 10 mM histidine, 8.5% sucrose, 0.04% PS20, 20 ug/mL EDTA, pH 5.6. A total of 16 monkeys were dosed with the anti-CD154 antibody C4LB231, 8 monkeys were dosed with the positive control antibody, and 4 monkeys were administered control vehicle.

TABLE 1
Experimental Design of 8-week Dose Study in Rhesus Monkeys

| Group | Test | Dose Level | Dose | Number of Animals | |
|---|---|---|---|---|---|
| No. | Material | (mg/kg/dose) | Route¹ | Male | Female |
| 1 | Control | 0 | IV | 2 | 2 |
| | vehicle | | | | |
| 2 | Positive | 50 | IV | 4 | 4 |
| | control | | | | |
| | antibody | | | | |
| 3 | C4LB231 | 150 | IV | 4 | 4 |
| 4 | C4LB231 | 30 | IV | 2 | 2 |
| 5 | C4LB231 | 100 | SC | 2 | 2 |

¹IV = intravenous slow bolus injection;
SC = subcutaneous injection

Groups 1-4 were administered control vehicle, positive control antibody, or test antibody C4LB231 via slow bolus IV injection into a peripheral vein and Group 5 was administered test antibody C4LB231 via SC injection in the scapular area once weekly on days 1, 8, 15, 22, 29, 36, 43, and 50 for a total of 8 doses. Blood samples were collected from the animals and evaluated as described below.

Results

Treatment with C4LB231 at 30 mg/kg IV, 150 mg/kg IV, and 100 mg/kg SC resulted in no thrombosis in any of the IV or SC dosed animals as determined by lung histopathology evaluation. No effects on coagulation panel (prothrombin time (PT), activated partial thromboplastin time (aPTT), fibrinogen (fib) and d-dimer (fibrin degradation product)) and no changes in platelet counts and morphology parameters (mean volume, component, and distribution width) were observed in the high dose group (150 mg/kg/wk IV). C4LB231-related changes in clinical pathology parameters were limited to moderately decreased lymphocytes for individual females at 150 mg/kg IV dose at day 56. No C4LB231 related injection or injection site reactions were observed. No other adverse effects were observed for C4LB231 doses up to 150 mg/kg.

Serum from the collected blood samples was analyzed for determination of serum concentration of C4LB231. The mean serum concentrations of C4LB231 determined for each antibody-dosed group is shown in Table 2 below. All C4LB231-dosed animals had quantifiable antibody concentrations throughout the sample collection period (day 1 to day 57) following weekly IV or SC administrations. All positive control antibody-dosed animals also had quantifiable serum concentrations throughout the sample collection period following weekly IV administrations. C4LB231 exposure at the high dose (150 mg/kg) exceeded that of the positive control antibody at 50 mg/kg.

TABLE 2
Mean Serum Estimates Following Eight Weekly IV/SC Administrations in
Rhesus Monkeys

| | Following Dose on Day 1 | | | Following Dose on Day 50 | | | |
|---|---|---|---|---|---|---|---|
| Group | Cₘₐₓ | Tₘₐₓ | AUC_{day1-8} | Cₘₐₓ | Tₘₐₓ | AUC_{day50-57} | |
| No. | (μg/mL) | (day)¹ | (μg * day/mL) | (μg/mL) | (day) | (μg * day/mL) | R² |
| 2 | 1030.97 | — | 4409.80 | 2892.38 | — | 16177.19 | 3.68 |
| 3 | 2968.08 | — | 8686.17 | 4882.18 | — | 23785.24 | 2.80 |
| 4 | 577.08 | — | 1510.90 | 1095.89 | — | 3089.42 | 2.10 |
| 5 | 1659.32 | 3.00 | 7891.58 | 2101.95 | 1.5 | 12118.30 | 1.66 |

¹Time elapsed from the given dose
²mean of individual ratios

Mean free and total sCD154 concentrations were determined in platelet poor plasma samples by electrochemiluminescence immunoassay (ECLIA). Platelet poor plasma samples were used because platelets shed CD154 creating difficulties in measuring sCD154 concentration. Mean free and total sCD154 concentrations are shown in FIGS. 1A and 1B. Mean free sCD154 concentrations were immediately suppressed below or around the lowest quantifiable concentration after the first dose and remained at this level following weekly IV doses of 50 mg/kg of the positive control antibody or 150 mg/kg of C4LB231 until the last sample was collected 7 days post the final antibody dose, with the exception of one animal which had free sCD154 baseline concentration below the lowest quantifiable limit.

Following administration of 30 mg/kg of C4LB231 on day 1 and day 50, mean free sCD154 concentrations at 1 hour post-dose were at or below the lowest quantifiable concentration and the mean sCD154 concentrations remained at least 50% lower than baseline at the end of each dose interval. Following SC administration of 100 mg/kg of C4LB231 on day 1 and day 50, mean free sCD154 concentrations at 6 hours post-dose were suppressed below the lowest quantifiable concentration and the mean sCD154 concentrations remained at the lowest quantifiable concentration level throughout the sampling period.

In Group 1, total plasma sCD154 concentrations in most samples were below or around the lowest quantifiable concentration. Following weekly IV doses of 30 and 150 mg/kg, or weekly SC doses of 100 mg/kg of C4LB231 as well as IV weekly doses of 50 mg/kg positive control antibody, total sCD154 concentrations immediately increased and reached a plateau before day 22. The total sCD154 concentration in the positive control antibody group reached a plateau earlier than the C4LB231 dosed groups. Mean plateau concentrations in the 150 mg/kg C4LB231 IV dose group were similar to the mean plateau concentrations in the 100 mg/kg C4LB231 SC dose group. Mean plateau concentrations in the 30 mg/kg C4LB231 IV dose group were slightly lower than the 150 mg/kg IV or 100 mg/kg SC C4LB231 dose groups. Mean plateau concentrations in the 50 mg/kg positive control antibody IV dose group were significantly lower.

Anti-KLH Antigen Challenge Study

Primary and secondary humoral immune responses were evaluated via detection of anti-keyhole limpet hemocyanin (KLH) antibodies after immunization on days 15 and 36. Animals were immunized with a dose of 1 mg of KLH via intramuscular injection at the posterior aspect of the left thigh. Blood samples were collected and anti-KLH IgG and IgM antibody analyses were conducted using a colorimetric enzyme-linked immunosorbent assay (ELISA). The results are shown in FIGS. 2A and 2B.

All animals in the control group generated robust/marked primary anti-KLH IgM and IgG antibody responses as demonstrated by the presence of center point titer (CPT) values for all control animals at 7, 14, and 21 days post primary immunization (IgM and IgG CPT reached group mean values of approximately 300 and 1400, respectively), and increased group mean values after secondary immunization with KLH on Day 36 (CPT group mean values up to approximately 600 and 3600 for IgM and IgG, respectively). Dose-dependent reductions in anti-KLH IgM and IgG primary and secondary responses were present for test antibody C4LB231 and positive control antibody-dosed animals. These reductions in anti-KLH antibody responses were an anticipated pharmacological effect of the administration of both antibodies. In sum, KLH challenges showed robust responses from control animals but dose dependent suppression of responses in C4LB231 treated groups, as expected.

II. 3-Month Dose Study

Rhesus monkeys were administered the anti-CD154 antibody C4LB231 intravenously (IV) or subcutaneously (SC) once weekly for 13 weeks. The experimental design of the study is shown in Table 3 below. The control vehicle was 0.9% sterile saline (NaCl). Animals were dosed by IV or SC injection as described above for the 8-week study on days 1, 8, 15, 22, 29, 36, 43, 50, 57, 64, 71, 78, and 85 for a total of 13 doses. On days 29, 57, and 155 (recovery study animals only) animals received an intramuscular injection of a 1 mg dose of KLH antigen to the left thigh for a KLH-antigen challenge study. Blood samples were collected from the animals and evaluated as described below.

TABLE 3
Experimental Design of 3-month Dose Study in Rhesus Monkeys

| | | | | Number of Animals² | |
|---|---|---|---|---|---|
| Group | Test | Dose Level | Dose | Main | Recovery |
| No. | Material | (mg/kg/dose) | Route¹ | Study | Study |
| 1 | Control | 0 | IV/SC | 6 | 4 |
| | vehicle | | | | |
| 2 | C4LB231 | 20 | IV | 6 | 4 |
| 3 | C4LB231 | 150 | SC | 6 | 4 |
| 4 | C4LB231 | 150 | IV | 6 | 4 |

¹IV = intravenous slow bolus injection; SC = subcutaneous injection
²Main study animals went to necropsy on day 92; recovery study animals went to necropsy on day 185

Results

No adverse effects were observed for treatment with C4LB231 up to 150 mg/kg/wk administered by IV or SC injection for the 13-week treatment period. No thrombosis as determined by lung histopathology evaluation and no effects on coagulation parameters were observed for the treatment groups, similar to the results observed in the 8-week dose study.

Serum from the collected blood samples was analyzed for determination of serum concentration of antibodies. The mean serum C4BL231 toxicokinetic (TK) parameter estimates following weekly IV doses in rhesus monkeys are summarized in Table 4 below. All C4BL231-treated animals had quantifiable serum C4BL231concentrations throughout the sample collection period following weekly IV or SC administration. Exposure increased in an approximately dose-proportional manner following the first IV dose on Day 1 and in a slightly greater than dose-proportional manner following the IV dose on Day 85. Serum concentrations reached the steady state by Day 92. Serum concentrations were quantifiable throughout the recovery period. Serum C4BL231 concentrations were below the lowest quantifiable limit through the sampling period in all animals from the control group (Group 1). Five out of the thirty animals treated with C4LB231 tested positive for anti-C4BL231 antibodies (i.e., anti-drug antibody (ADA)-positive). Mean T_{1/2} values estimated in the ADA-negative recovery animals were similar across different dose groups and there were no apparent differences between male and female animals.

TABLE 4
Mean Serum Estimates Following Thirteen Weekly IV/SC Administrations in
Rhesus Monkeys

| | Following Dose on Day 1 | | | Following Dose on Day 85 | | | | |
|---|---|---|---|---|---|---|---|---|
| Group | Cₘₐₓ | Tₘₐₓ | AUC_{day1-8} | Cₘₐₓ | Tₘₐₓ | AUC_{day50-57} | | |
| No. | (μg/mL) | (day)¹ | (μg * day/mL) | (μg/mL) | (day) | (μg * day/mL) | R² | T_{1/2}³ |
| 2 | 433.83 ± | — | 1118.44 ± | 585.99 ± | — | 2036.34 ± | 1.80 ± | 17.60 ± |
| | 22.41 | | 178.14 | 105.65 | | 790.83 | 0.61 | 3.10 |
| 3 | 1092.66 ± | 2.5 ± | 6282.83 ± | 2809.82 ± | 1.30 ± | 16951.90 ± | 2.71 ± | 15.52 ± |
| | 169.75 | 1.58 | 894.69 | 384.30 | 0.95 | 3023.50 | 0.35 | 2.06 |
| 4 | 3015.89 ± | — | 9663.44 ± | 5172.64 ± | — | 21203.51 ± | 2.18 ± | 15.59 ± |
| | 307.30 | | 910.66 | 1112.16 | | 6065.53 | 0.49 | 0.61 |

¹time elapsed from the given dose
²mean of individual ratios
³ADA-negative recovery animals only [Group 2 N = 3; Group 3 N = 4; Group 4 N = 3]

Free and total soluble CD154 (sCD154) were assessed in platelet poor plasma samples. The data are shown in FIGS. 1C and 1D. Free sCD154 concentrations were immediately reduced following the IV or SC dosing of C4BL231. The extent of reduction was greater at the higher dose levels. A dose-dependent decrease in free sCD154 coupled with dose-dependent increase in total sCD154 indicated good target engagement in plasma.

C4BL231-related, dose-dependent changes in the production of anti-KLH IgM and IgG antibodies was observed in both 150 mg/kg/dose groups (IV and SC) relative to control (Group 1) beginning at 14 days post-primary immunization, and for all groups (including 20 mg/kg/dose; Group 2), and at all time points postsecondary immunization with KLH. This change was more distinct in response post-secondary immunization with KLH. This decrease in anti-KLH IgM and IgG antibodies was also present at 20 mg/kg (Group 2) for some individual female animals, and some individual male and female animals at 150 mg/kg (Groups 3 and 4) post-tertiary immunization on Day 155 during the recovery period which correlated with quantifiable serum C4LB231 concentrations for the same individual animals. Given these results, a 3-month recovery period was not sufficient to restore the full KLH antibody response following C4BL231 administration. The decreased primary and secondary anti-KLH IgM and IgG antibody responses were an anticipated pharmacological effect of C4BL231administration.

In sum, administration of C4BL231 by IV or SC injection once weekly was well tolerated in rhesus monkeys at levels up to 150 mg/kg (SC and IV) for 3 months. Some treatment-related findings were observed at high dose groups of 150 mg/kg IV or SC (e.g., decreased lymphocytes and monocytes; reduced number and size of lymphoid follicles and germinal centers in the spleen, mandibular and mesenteric lymph nodes; and apparent suppression of T-cell-dependent antibody responses (TDAR)). However, such treatment-related findings were likely the results of the intended pharmacology of CD154 antagonism. Based on these results, the no-observed adverse effect level (NOAEL) was considered to be 150 mg/kg/week IV and SC corresponding to Cₘₐₓ at 5172.6 and 2809.8 μg/mL for IV and SC administration, respectively, and AUC_{day85-92} at 21203.5 and 16951.9 days*μg/mL for IV and SC administration, respectively.

III. 6-Month Dose Study

Rhesus monkeys are administered the anti-CD154 antibody C4LB231 subcutaneously (SC) once weekly for 6 months. The experimental design of the study is shown in Table 5 below. The control vehicle is 0.9% sterile saline (NaCl). Animals are dosed on days 1, 8, 15, 22, 29, 36, 43, 50, 57, 64, 71, 78, 85, 92, 99, 106, 113, 120, 127, 134, 141, 148, 155, 162, 169, and 176 for a total of 26 doses.

TABLE 5
Experimental Design of 6-month Dose Study in Rhesus Monkeys

| | | | | Number of Animals | |
|---|---|---|---|---|---|
| Group | Test | Dose Level | Dose | Main | Recovery |
| No. | Material | (mg/kg/dose) | Route¹ | Study | Study |
| 1 | Control | 0 | SC | 6 | 4 |
| | vehicle | | | | |
| 2 | C4LB231 | 30 | SC | 6 | 4 |
| 3 | C4LB231 | 150 | SC | 6 | 4 |

¹SC = subcutaneous injection

Blood samples are collected for determination of serum concentrations of C4BL231 and anti-C4BL231 antibody (i.e., anti-drug antibody (ADA)) analysis. Other analysis (e.g., determination of sCD154 concentration) may be performed and other studies of the animals (e.g., KLH antigen challenge studies) may be conducted to further evaluate safety of C4BL231 administration.

The study is ongoing. No notable clinical observations have been made to date in either the 30 mg/kg or 150 mg/kg treatment groups.

### Example 2

### Clinical Study to Evaluate Safety and Tolerability of Parenterally Administered Anti-CD154 Antibody

A randomized, double-blind, placebo-controlled study in healthy male and female participants aged 18-55 years old was conducted. The 56 enrolled participants were randomly divided into seven cohorts with eight participants each. Five of the cohorts were intravenously administered the anti-CD154 antibody C4LB231 at a dosage of 0.3 mg/kg, 1 mg/kg, 3 mg/kg, 10 mg/kg, or 30 mg/kg; and one cohort was subcutaneously administered C4LB231 at a dosage of 3 mg/kg. Initially, a sixth cohort was scheduled to be intravenously administered the anti-CD154 antibody at a dosage of 50 mg/kg. However, sufficient pharmacodynamic effects were observed for the cohort dosed with 30 mg/kg antibody, so the decision was made to not escalate the study to the 50 mg/kg cohort (see Example 3 below). Within each cohort, six participants were randomized to receive the C4LB231 antibody and two participants were randomized to receive placebo. Sentinel dosing of each cohort was performed: the first two subjects (one active and one placebo) in each cohort were dosed at least 24 hours before the other subjects were dosed. Infusions were administered with the use of a syringe pump for the lower dose groups (0.3 mg/kg to 3 mg/kg) whereas IV bags and volumetric pumps were used for the higher dosage groups (10 mg/kg to 30 mg/kg).

Study participants in the intravenous cohorts were administered a total dosage of 0.3 mg/kg, 1 mg/kg, 3 mg/kg, 10 mg/kg, or 30 mg/kg by intravenous infusion of antibody diluted to a volume of up to 250 mL over a period of about 60 minutes to 120 minutes. Study participants in the subcutaneous cohort were administered a total dosage of 3 mg/kg antibody. The total dosage was administered in up to four (4) subcutaneous injections (depending on body weight) in the abdominal area. The intravenous cohorts were administered at escalating dosage levels, meaning that the 0.3 mg/kg cohort was dosed first, and the decision to proceed with administration of the next highest dosage level was made after safety evaluation of clinical and laboratory data from the previously dosed cohort, including pharmacokinetic and pharmacodynamics data, data from antigen-challenge tests (e.g., tetanus toxoid and KLH), viral levels (e.g., EBV and CMV), etc. The duration of the study is about 20 weeks, including a screening period of four weeks prior to administration of the study formulation and a 16-week follow-up period. The study is ongoing.

Safety and tolerability of C4LB231 was monitored by physical examinations, assessment of vital signs (e.g., temperature, pulse/heart rate, respiratory rate, blood pressure, etc.), electrocardiogram, cardiac telemetry, clinical laboratory tests, thrombosis laboratory assessments, early detection active tuberculosis (TB) assessments, concomitant medications, and reported adverse events and serious adverse events, including infusion and allergic reactions and injection site reactions.

Blood samples were collected from the participants for pharmacokinetic (PK), pharmacodynamics (PD), and immunogenicity assessments, as discussed in the examples below. Blood samples were also collected for additional biomarker analyses including assessment of proinflammatory cytokines (in serum); and immunophenotyping of lymphocyte subsets as well as T- and B-cell functional analyses in peripheral blood mononuclear cells (PBMCs). As the study is ongoing, samples are still being collected from study participants and analyzed. The data obtained thus far indicate no safety signals for administration of C4BL231 (e.g., no severe adverse effects (SAEs), no thromboembolic effect or trends in coagulation safety parameters, and no cytomegalovirus (CMV) or Epstein Barr virus (EBV) reactivation).

### Example 3

### Pharmacokinetics Evaluation

Plasma samples were obtained from the study subjects at various time points following administration of C4LB231 according to the study described in Example 2. Pharmacokinetic parameters of C4LB231 were subsequently determined from plasma concentrations in subjects over time subsequent to administration of C4LB231. The pharmacokinetic parameters determined include Cₘₐₓ (maximum observed plasma concentration), Tₘₐₓ (time to reach maximum observed plasma concentration); AUC_{inf} (area under plasma concentration versus time curve from time zero to infinity with extrapolation of the terminal phase); AUCₗₐₛ (area under plasma concentration versus time curve from time zero to the time corresponding to the last quantifiable concentration); T_{1/2} (terminal half-life); CL (total systemic clearance, for IV administration); CL/F (apparent total systemic clearance after extravascular administration, for SC administration); V_{z} (volume of distribution based on terminal phase, for IV administration); V_{z}/F (apparent volume of distribution based on terminal phase after extravascular administration, for SC only); and F(%) (absolute SC bioavailability calculated by (AUC_{inf,SC}/mean AUC_{inf, IV})*100).

Preliminary pharmacokinetic analyses are shown in FIGS. 3A-3D. The data indicate a dose proportional increase in Cₘₐₓ from 0.3 mg/kg to 30 mg/kg, and an approximate dose proportional increase in AUC from 0.3 mg/kg to 10 mg/kg. Greater than dose proportional increases in AUC between 10 mg/kg and 30 mg/kg were observed. The half-life (t_{1/2}) of C4BL231 is preliminarily determined to be about 7 to 11 days (at the time, not enough data is available to determine t_{1/2} for the 30 mg/kg cohort; to be updated later). Based on the currently available data, bioavailability (F %) for SC administration is determined to be 71%.

Based on the Cₘₐₓ data thus far, a sufficient safety margin is predicted even at a dose of 50 mg/kg if escalated. Based on the most conservative AUC safety margin (AUC over 1 dosing interval from the 3-month dose toxicity study in rhesus monkeys described above in Example 1), the AUCs in the study described in Example 2 were higher. However, based on another acceptable method of determining the AUC safety margin using the cumulative AUC from the 3-month dose toxicity study in rhesus monkeys described above in Example 1, there was also a sufficient safety margin. However, since sufficient pharmacodynamic (PD) effects were observed (e.g., soluble CD154, anti-KLH and anti-tetanus responses as described below) this warranted stopping at the 30 mg/kg cohort. Thus, the decision was made to not escalate the study to the 50 mg/kg cohort.

### Example 4

### Clinical Study to Evaluate Primary and Recall Immune Responses to Antigen Challenges

Participants of the clinical study described in Example 2 were administered keyhole limpet hemocyanin (KLH) antigen to assess primary immune response and tetanus toxoid (TT) to assess recall (memory) immune response.

KLH Antigen Challenge

Participants of the clinical study described in Example 2 were administered a single intramuscular injection of keyhole limpet hemocyanin (KLH) reconstituted in an aluminum hydroxide based-adjuvant (Immuncothel®) about three days after administration of C4LB231. The total dosage of reconstituted KLH administered was 3 mg. The intramuscular injection is administered in either the deltoid muscle or the lateral mid-thigh.

Tetanus Toxoid Antigen Challenge

Participants of the clinical study described in Example 2 who demonstrated serologic evidence of pre-existing protective immunity against tetanus prior to being selected for participation in the study were administered a single intramuscular injection of commercially available tetanus toxoid (0.5 units) about 3 days after administration of C4LB231. The intramuscular injection was administered in either the deltoid muscle or lateral mid-thigh, but only one injection per location was administered (i.e., KLH antigen in the deltoid and tetanus toxin in the thigh, or vice versa).

Serum samples were collected from participants for analysis of the immune response to KLH and tetanus toxoid, including anti-KLH IgG, anti-KLH IgM, and anti-tetanus IgG levels. Human anti-KLH antibodies in serum/plasma were measured using the ELISA method described in Aarntzen et. al. Cancer Immunol. Immunother. (2012) 61(11): 2003-11. Preliminary data is shown in FIGS. 4A-4D. Treatment with C4LB231 resulted in near total inhibition of the anti-KLH response (primary response) at doses ≥3 mg/kg IV. Treatment with C4LB231 resulted in inhibition of anti-tetanus toxoid response (secondary or recall response) at doses ≥10 mg/kg IV. These data indicate that administration of C4BL231 at 3 mg/kg IV inhibits primary antibody response to KLH antigen, but administration of ≥10 mg/kg C4BL231 IV may be needed to impact recall response to tetanus toxoid. This suggests that secondary (recall) immune response may require a higher dose than that needed to inhibit primary immune response, and that blocking CD154 leads to reduced T cell: B cell interactions resulting in lower humoral response to antigen stimulus.

### Example 5

### Immunogenicity Evaluation

Antibodies to C4LB231 are evaluated in serum samples collected from participants administered C4LB231 according to the study described in Example 1. Samples derived from C4LB231 dosed subjects will be screened for antibodies binding C4LB231, if positive, the specificity and titer of samples that test positive for anti-C4LB231 antibodies will be determined. Participants are classified as positive for antibodies to C4LB231 if any posttreatment samples test positive for anti-C4LB231 antibodies; and participants are classified as negative for antibodies to C4LB231 if anti-C4LB231 antibodies are not detected in any posttreatment samples. Based upon the individual subject pharmacokinetic profiles described in Example 3 and shown in FIGS. 2A and 2B, there is no obvious evidence of anti-C4LB231 antibodies in the samples obtained from the C4LB231 dosed subjects; sample analysis is ongoing.

### Example 6

### Evaluation of Total and Free Plasma Soluble CD154

Mean free and total soluble CD154 (sCD154) concentrations were determined by ELISA in platelet poor plasma samples obtained from the participants administered C4LB231 according to the study described in Example 2. Preliminary data obtained thus far are shown in FIGS. 5A-5B. Free sCD154 concentration remained undetectable until 14 days after dosing indicating suppression of the target of C4BL231. Total sCD154 concentration correspondingly increased until reaching a peak value and declined thereafter over time.

### Example 7

### Platelet Activation Study

Platelet activation studies were conducted to further evaluate the mechanism of platelet activation by anti-CD154 antibodies. Blood from healthy human donors was collected. Platelet activation was evaluated by flow cytometry using validated platelet activation markers PAC-1 (activated GPIIb/IIIa) and CD62p (P-selectin). Briefly, whole blood (WB) was added to buffer and anti-PAC1 and anti-CD62p antibodies with or without anti-FcγRIIa antibody were added to the mixture and incubated for 25 minutes. Pre-formed complexes of sCD154 and antibody at a molar ratio of 3:1 CD154: anti-CD154 were added to the mixture, or antibody alone was added to the mixture, and incubated for another 20 minutes. Platelets were fixed in 1% formalin followed by FACS analysis. The results are shown in FIGS. 6A and 6B.

The results show that platelet activation was induced by sCD154-“BG9588” (sCD154-5c8IgG1). In contrast, platelet activation was not induced by sCD154 in combination with either Fc-silent C4BL231 or wild-type-Fc tail (which is capable of platelet Fc-binding) activated platelets. None of the antibodies alone activated platelets (“no soluble CD154”). Additional constructs having C4LB231 variable region on various Fcs (including IgG1) alone or complexed with sCD154 also did not induce platelet activation (FIG. 6B). This suggests that an active Fc domain may not be the sole determinant of platelet activation by anti-CD154 antibodies. Rather, the data indicate that some antibodies having a silent Fc are capable of platelet activation leading to the conclusion that both the variable domains and the higher order antibody/sCD154 complex formation contribute to platelet activation.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

### REFERENCES

1. WO2017/024146
2. Alaaeddine N, Hassan G S, Yacoub D, Mourad W. CD154: an immunoinflammatory mediator in systemic lupus erythematosus and rheumatoid arthritis. Clin Dev Immunol. 2012; 2012: 1-11 doi:10.1155/2012/490148.
3. Albulescu M G, Gunsior M, Li J, et al. Safety, pharmacokinetics, pharmacodynamics and inhibition of T-cell dependent antibody response (TDAR) With MEDI4920, a novel, engineered CD40 ligand (CD40L) antagonist: results of a first-time-in-human study. Scientific Abstracts. 2017; 17 Jun.:867-868. DOI: 10.1136/annrheumdis-2017-eular.3468.
4. American Red Cross. http://www.redcrossblood.org/donating-blood/donation-faqs. Accessed 8 Dec. 2016.
5. Bazett H C. An analysis of the time-relationship of electrocardiograms. Heart. 1920; 7:353-380.
6. Berner B, Wolf G, Hummel K M, Muller G A, Reuss-Borst M A. Increased expression of CD40 ligand (CD154) on CD4+ T cells as a marker of disease activity in rheumatoid arthritis. Ann Rheum Dis. 2000; 59(3):190-195.
7. Boulton C, Meiser K, David O J, Schmouder R. Pharmacodynamic effects of steady-state fingolimod on antibody response in healthy volunteers: a 4-week, randomized, placebo-controlled, parallel-group, multiple-dose study. J Clin Pharmacol. 2012; 52(12):1879-1890. doi: 10.1177/0091270011427908. Epub 2011 Dec. 15.
8. Boumpas D T, Furie R, Manzi S, Illei G G, Wallace D J, Balow J E, Vaishnaw A; BG9588 Lupus Nephritis Trial Group. A short course of BG9588 (anti-CD40 ligand antibody) improves serologic activity and decreases hematuria in patients with proliferative lupus glomerulonephritis. Arthritis Rheum. 2003; 48(3):719-727.
9. Chamberlain C, Colman P J, Ranger A M, et al. Repeated administration of dapirolizumab pegol in a randomised phase I study is well tolerated and accompanied by improvements in several composite measures of systemic lupus erythematosus disease activity and changes in whole blood transcriptomic profiles. Ann Rheum Dis. 2017; 76:1837-1844.
10. Committee for Medicinal Products for Human Use (CHMP). Guideline on requirements for first-in-man clinical trials for potential high-risk medicinal products. EMEA. 2007; 1-11. Doc.RefEMEA/CHMP/SWP/28367/2007 Corr.
11. Couzin J. Magnificent Obsession. Science 2005; 307:1712-1715.
12. Davis J C. Totoritis M C, Rosenberg J, et al. Phase I clinical trial of a monoclonal antibody against CD40-ligand (IDEC-131) in patients with systemic lupus erythematosus. J Rheumat. 2001; 28:95-101.
13. Dewitte A, Tanga A, Villeneuve J, et al. New frontiers for platelet CD154. Experimental Hematology & Oncology. 2015; 4:6. https://doi.org/10.1155/2017/2982879.
14. Dumont F J. IDEC-131 IDEC/Eisai.Current Opinion in Investigational Drugs. 2002; 3(5):725-734.
15. Elgueta R, Benson M J, de Vries V C, Wasiuk A, Guo Y, Noelle R J. Molecular mechanism and function of CD40/CD40L engagement in the immune system. Immunological reviews. 2009; 229(1):152-172.
16. EMEA. Guideline on strategies to identify and mitigate risks for first-in-human and early clinical trials with investigational medicinal products EMEA/CHMP/SWP/28367/07 Rev. 1. http://www.ema.europa.eu/docs/en_GB/document_library/Scientific_guideline/2017/07/WC500232186.pdf. (accessed 9 Jan. 2018)
17. Ford M L, Adams A B, Pearson T C. Targeting co-stimulatory pathways: transplantation and autoimmunity. Nature Reviews Nephrology. 2014; 10: 14-24
18. Hassan G S, Yacoub D, Alaaeddine N, Nadiri A, Merhi Y, Mourad W. CD154: the atherosclerotic risk factor in rheumatoid arthritis? Arthritis Res Ther. 2013; 15(1):206.
19. Green J D. EUFEPS & EAPB Workshop on Monoclonal Antibodies, Amsterdam, Apr. 26-27, 2007.
20. International Conference on Harmonisation of Technical Requirements for Registration of Pharmaceuticals for Human Use. ICH Harmonized Tripartite Guideline E14: Clinical evaluation of QT/QTc interval prolongation and proarrhythmic potential for non-antiarrhythmic drugs. ICH 12 May 2005.
21. Kalunian K C, Davis J C, Merrill J T, et al. Treatment of Systemic lupus erythematosus by inhibition oft cell costimulation with anti-CD154. Arthritis and Rheumatism. 2002; 46(12):3251-3258.
22. Kappos L, Mehling M, Arroyo R, et al. Randomized trial of vaccination in fingolimod-treated patients with multiple sclerosis. Neurology. 2015; 84:872-879.
23. Kuwana M, Nomura S, Fujimura K, Nagasawa T, Muto Y, Kurata Y, Tanaka S, Ikeda Y. Effect of a single injection of humanized anti-CD154 monoclonal antibody on the platelet-specific autoimmune response in patients with immune thrombocytopenic purpura. Blood. 2004; 103(4):1229-1236
24. Li J, Gunsior M, Ly N, et al. Pharmacokinetics, Pharmacodynamics, and Immunogenicity of MEDI4920, a Novel, Engineered CD40 Ligand Antagonist, in Healthy Volunteers [abstract]. Arthritis Rheumatol. 2016; 68 (suppl 10). http://acrabstracts.org/abstract/pharmacokinetics-pharmacodynamics-andimmunogenicity-of-medi4920-a-novel-engineered-cd40-ligand-antagonist-in-healthyvolunteers/Accessed Oct. 17, 2017.
25. Milton M N and Horvath C J. The EMEA Guideline on First-in-Human Clinical Trials and Its Impact on Pharmaceutical Development. Toxicologic Pathology, 2009; 37:363-371.
26. Patel V, Schwartz J, Bussel J. The effect of anti-CD40 ligand in immune thrombocytopenic purpura. BJH. 2007; 141:545-548.
27. Poirier N, Mary C, Dilek N, et al. Preclinical efficacy and immunological safety of FR104, an antagonist anti-CD28 monovalent Fab′ antibody. Am J Transplant. 2012; (10):2630-40.
28. Rossini A A, Parker D C, Phillips N E, Durie F H, Noelle R J, Mordes J P, Greiner D L. Induction of immunological tolerance to islet allografts. Cell Transplant. 1996; 5(1):49-52.
29. Rosenzweig M, Rosenthal C A, torres V M, Vaickus L. Development of a quantitative assay to measure EBV viral load in patients with autoimmune type 1 diabetes and healthy subjects. J of Virological Methods. 2010; 164:111-115.
30. Shock A, Burkly L, Wakefield I, et al. CDP7657, an anti-CD40L antibody lacking an Fc domain, inhibits CD40L-dependent immune responses without thrombotic complications: an in vivo study. Arthritis Research & Therapy. 2015; 17(1):234. doi:10.1186/s13075-015-0757-4.
31. Swaminathan A, Lucas R M, Dear K, McMichael A J. Keyhole limpet haemocyanin—a model antigen for human immunotoxicological studies. Br J Clin Pharmacol. 2014; 78:1135-1142.
32. Tocoian A, Buchan P, Kirby H, Soranson J, Zamacona M, Walley R, Mitchell N, Esfandiari E, Wagner F, Oliver R. First-in-human trial of the safety, pharmacokinetics and immunogenicity of a PEGylated anti-CD40L antibody fragment(CDP7657) in healthy individuals and patients with systemic lupus erythematosus. Lupus. 2015 September; 24(10):1045-1056.
33. U.S. Department of Health and Human Services Food and Drug Administration Center for Drug Evaluation and Research (CDER). Guidance for Industry Estimating the Maximum Safe Starting Dose in Initial Clinical Trials for Therapeutics in Adult Healthy Volunteers. July 2005; Pharmacology and Toxicology:1-27.
34. U.S. Department of Health and Human Services Food and Drug Administration Center for Drug Evaluation and Research (CDER). Guidance for Industry Toxicity Grading Scale for Healthy Adult and Adolescent Volunteers Enrolled in Preventive Vaccine Clinical Trials. September 2007; Center for Biologics Evaluation and Research:1-8.
35. Verkruyse L A, Storch G A, Devine S M, et al. Once daily ganciclovir as initial pre-emptive therapy delayed until threshold CMV load X10000 copies/ml: a safe and effective strategy for allogeneic stem cell transplant patients. Bone Marrow Transplantation. 2006; 37:51-56.
36. Weinberg A, Boulware D, Dighero B, et al. Effect of abatacept on immunogenicity of vaccines in individuals with type 1 diabetes. Vacine. 2013; 31(42):4791-4794.
37. Yazdany J, Davis J. The role of CD40 ligand in systemic lupus erythematosus. Lupus. 2004; 13:377-380.
38. Barnes P F. Diagnosing latent tuberculosis infection: Turning glitter to gold [editorial]. Amer J Respir Crit Care Med. 2004; 170:5-6.
39. Brock I, Weldingh K, Lillebaek T, et al. Comparison of tuberculin skin test and new specific blood test in tuberculosis contacts. Am J Respir Crit Care Med. 2004; 170:65-69.
40. Cellestis. QuantiFERON-TB Gold clinicians guide and QuantiFERON-TB Gold In-Tube Method package insert. Downloaded from www.cellestis.com, February 2009.
41. Diel R, Loddenkemper R, Meywald-Walter K, Niemann S, Nienhaus A. Predictive value of a whole blood IFN-γ assay for the development of active tuberculosis disease after recent infection with mycobacterium tuberculosis. Am J Respir Crit Care Med. 2008; 177:1164-1170.
42. Ewer K, Deeks J, Alvarez L, et al. Comparison of T-cell-based assay with tuberculin skin test for diagnosis of Mycobacterium tuberculosis infection in a school tuberculosis outbreak. Lancet. 2003; 361:1168-73.
43. Ferrara G, Losi M, Meacci M, et al. Routine hospital use of a new commercial whole blood interferon-γ assay for the diagnosis of tuberculosis infection. Am J Respir Crit Care Med. 2005; 172:631-635.
44. Higuchi K, Nobuyuki H, Mori T, Sekiya Y. Use of QuantiFERON-TB Gold to investigate tuberculosis contacts in a high school. Respirology. 2007; 12:88-92.
45. Kobashi Y, Mouri K, Obase Y, et al. Clinical evaluation of QuantiFERON-TB-2G test for immunocompromised patients. Eur Respir J. 2007; 30:945-950.
46. Matulis G, Jüni P, Villiger P M, Gadola S D. Detection of latent tuberculosis in immunosuppressed patients with autoimmune diseases: performance of a Mycobacterium tuberculosis antigen-specific interferon γ assay. Ann Rheum Dis. 2008; 67:84-90
47. Mori T, Sakatani M, Yamagishi F, et al. Specific detection of tuberculosis infection: An interferon-γ-based assay using new antigens. Am J Respir Crit Care Med. 2004; 170:59-64.

1. A method of providing clinically proven safe administration of an anti-CD154 antibody or antigen binding fragment thereof comprising heavy chain complementarity determining regions (CDRs) HCDR1, HCDR2, and HCDR3 of SEQ ID NOs: 3, 4, and 5, respectively, and light chain CDRs LCDR1, LCDR2, and LCDR3, of SEQ ID NOs: 6, 7, and 8, respectively, to a human subject in need thereof, the method comprising subcutaneously or intravenously administering to the subject a pharmaceutical composition comprising the anti-CD154 antibody or antigen binding fragment thereof and a pharmaceutically acceptable carrier, wherein a total dosage of the anti-CD154 antibody or antigen binding fragment thereof administered is 0.3 mg/kg to 50 mg/kg body weight of the subject per administration.

2. The method of claim 1, wherein the total dosage of the anti-CD154 antibody or antigen binding fragment thereof administered is 0.3 mg/kg, 1 mg/kg, 3 mg/kg, 10 mg/kg, 30 mg/kg or 50 mg/kg, or any dosage in between.

3. The method of claim 1, wherein the pharmaceutical composition is administered subcutaneously.

4. The method of claim 3, wherein the total dosage of the anti-CD154 antibody or antigen binding fragment thereof is administered in one, two, three, or four subcutaneous injections per administration.

5. The method of claim 1, wherein the pharmaceutical composition is administered intravenously.

6. The method of claim 1, wherein the clinically proven safe administration of the anti-CD154 antibody or antigen binding fragment thereof comprises viremia of <10,000 copies of viral DNA of at least one virus selected from the group consisting of Epstein-Barr virus (EBV) and cytomegalovirus (CMV) per mL of sample from the subject.

7. The method of claim 1, wherein the clinically proven safe administration of the anti-CD154 antibody or antigen binding fragment thereof comprises an immune response comprising at least one of a recall response and a primary response, preferably an immune response comprising a recall response to tetanus toxoid and a primary response to keyhole limpet hemocyanin (KLH).

8. The method of claim 1, wherein the administration of the anti-CD154 antibody or antigen binding fragment thereof does not result in any clinically apparent thromboembolic (TE) event in the subject.

9. The method of claim 1, wherein the administration of the anti-CD154 antibody or antigen binding fragment thereof does not result in activation of platelets.

10. A method of providing clinically proven safe treatment of an autoimmune disease in a human subject in need thereof, the method comprising subcutaneously or intravenously administering to the subject a pharmaceutical composition comprising an anti-CD154 antibody or antigen binding fragment thereof comprising heavy chain complementarity determining regions (CDRs) HCDR1, HCDR2, and HCDR3 of SEQ ID NOs: 3, 4, and 5, respectively, and light chain CDRs LCDR1, LCDR2, and LCDR3, of SEQ ID NOs: 6, 7, and 8, respectively, and a pharmaceutically acceptable carrier, wherein a total dosage of the anti-CD154 antibody or antigen binding fragment thereof administered is 0.3 mg/kg to 50 mg/kg body weight of the subject per administration.

11. The method of claim 10, wherein the total dosage of the anti-CD154 antibody or antigen binding fragment thereof administered is 0.3 mg/kg, 1 mg/kg, 3 mg/kg, 10 mg/kg, 30 mg/kg or 50 mg/kg or any dosage in between.

12. The method of claim 10, wherein the pharmaceutical composition is administered subcutaneously.

13. The method of claim 12, wherein the total dosage of the anti-CD154 antibody or antigen binding fragment thereof is administered in one, two, three, or four subcutaneous injections per administration.

14. The method of claim 10, wherein the pharmaceutical composition is administered intravenously.

15. The method of claim 10, wherein the autoimmune disease is rheumatoid arthritis, systemic lupus erythematosus (SLE), or Sjögren's Syndrome.

16. The method of claim 10, wherein the clinically proven safe treatment of the anti-CD154 antibody or antigen binding fragment thereof comprises viremia of <10,000 copies of viral DNA of at least one virus selected from the group consisting of Epstein-Barr virus (EBV) and cytomegalovirus (CMV) per mL of sample from the subject.

17. The method of claim 10, wherein the clinically proven safe treatment of the anti-CD154 antibody or antigen binding fragment thereof comprises an immune response comprising at least one of a recall response and a primary response, preferably an immune response comprising a recall response to tetanus toxoid and a primary response to keyhole limpet hemocyanin (KLH).

18. The method of claim 10, wherein the administration of the anti-CD154 antibody or antigen binding fragment thereof does not result in any clinically apparent thromboembolic (TE) event in the subject.

19. The method of claim 10, wherein the administration of the anti-CD154 antibody or antigen binding fragment thereof does not result in activation of platelets.

20. The method of claim 1, wherein the pharmaceutical composition comprises 40 mg/mL to 60 mg/mL of the anti-CD154 antibody or antigen binding fragment thereof, 1 mM to 20 mM histidine, 5% to 10% (w/v) sucrose, 0.01% to 0.10% (w/v) polysorbate 20 (PS20), and 10 μg/mL to 30 μg/mL EDTA, at pH 5.0-6.0.

21. The method of claim 1, wherein the pharmaceutical composition comprises 40 mg/mL to 60 mg/mL of the anti-CD154 antibody or antigen binding fragment thereof, 1 mM to 20 mM arginine, 5% to 10% (w/v) lactose, 0.01% to 0.10% (w/v) polysorbate 80 (PS80), and 10 μg/mL to 30 μg/mL EDDS, at pH 5.0-6.0.

22. The method of claim 1, wherein the pharmaceutical composition comprises 40 mg/mL to 60 mg/mL of the anti-CD154 antibody or antigen binding fragment thereof, 1 mM to 20 mM glycine, 5% to 10% (w/v) maltose, 0.01% to 0.10% (w/v) polysorbate 80 (PS80), and 10 μg/mL to 30 μg/mL EDTA, at pH 5.0-6.0.


SEQUENCE LISTING (12 sequences)

SEQ ID NO: 1 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 2 - protein, 149 aa, Homo sapiens
MQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 3 - protein, 5 aa, Artificial Sequence
  C4LB231 HCDR1
SYGIS

SEQ ID NO: 4 - protein, 17 aa, Artificial Sequence
  C4LB231 HCDR2
WISPIFGNTNYAQKFQG

SEQ ID NO: 5 - protein, 9 aa, Artificial Sequence
  C4LB231 HCDR3
SRYYGDLDY

SEQ ID NO: 6 - protein, 11 aa, Artificial Sequence
  C4LB231 LCDR1
RASQSISSYLN

SEQ ID NO: 7 - protein, 7 aa, Artificial Sequence
  C4LB231 LCDR2
YANSLQS

SEQ ID NO: 8 - protein, 9 aa, Artificial Sequence
  C4BL231 LCDR3
QQSDSIPWT

SEQ ID NO: 9 - protein, 118 aa, Artificial Sequence
  C4BL231 VH
QVQLVQSGAEVKKPGSSVKVSCKASGGTFSSYGISWVRQAPGQGLEWMGWISPIFGNTNYAQKFQGRVTITADESTSTAYMELSSLRSEDTAVYYCARSRYYGDLDYWGQGTLVTVSS

SEQ ID NO: 10 - protein, 107 aa, Artificial Sequence
  C4BL231 VL
DIQMTQSPSSLSASVGDRVTITCRASQSISSYLNWYQQKPGKAPKLLIYYANSLQSGVPSRFSGSGSGTDFTLTISSLQPEDFATYYCQQSDSIPWTFGQGTKVEIK

SEQ ID NO: 11 - protein, 448 aa, Artificial Sequence
  C4BL231 Heavy Chain
QVQLVQSGAEVKKPGSSVKVSCKASGGTFSSYGISWVRQAPGQGLEWMGWISPIFGNTNYAQKFQGRVTITADESTSTAYMELSSLRSEDTAVYYCARSRYYGDLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 214 aa, Artificial Sequence
  C4BL231 Light Chain
DIQMTQSPSSLSASVGDRVTITCRASQSISSYLNWYQQKPGKAPKLLIYYANSLQSGVPSRFSGSGSGTDFTLTISSLQPEDFATYYCQQSDSIPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC